SEQUENCE LISTING (21 sequences)

SEQ ID NO: 1 - DNA, 1422 bp, Marinobacter hydrocarbonoclasticus
atgaaacgtctcggaaccctggacgcctcctggctggcggttgaatctgaagacaccccgatgcatgtgggtacgcttcagattttctcactgccggaaggcgcaccagaaaccttcctgcgtgacatggtcactcgaatgaaagaggccggcgatgtggcaccaccctggggatacaaactggcctggtctggtttcctcgggcgcgtgatcgccccggcctggaaagtcgataaggatatcgatctggattatcacgtccggcactcagccctgcctcgccccggcggggagcgcgaactgggtattctggtatcccgactgcactctaaccccctggatttttcccgccctctttgggaatgccacgttattgaaggcctggagaataaccgttttgccctttacaccaaaatgcaccactcgatgattgacggcatcagcggcgtgcgactgatgcagagggtgctcaccaccgatcccgaacgctgcaatatgccaccgccctggacggtacgcccacaccaacgccgtggtgcaaaaaccgacaaagaggccagcgtgcccgcagcggtttcccaggccatggacgccctgaagctccaggcagacatggcccccaggctgtggcaggccggcaatcgcctggtgcattcggttcgacacccggaagacggactgaccgcgcccttcactggaccggtttcggtgctcaatcaccgggttaccgcgcagcgacgttttgccacccagcattatcaactggaccggctgaaaaacctggcccatgcttccggcggttccttgaacgacatcgtgctttacctgtgtggcaccgcattgcggcgctttctggctgagcagaacaatctgccagacaccccgctgacggctggtataccggtgaatatccggccggcagacgacgagggtacgggcacccagatcagtttcatgattgcctcgctggccaccgacgaagctgatccgttgaaccgcctgcaacagatcaaaacctcgacccgacgggccaaggagcacctgcagaaacttccaaaaagtgccctgacccagtacaccatgctgctgatgtcaccctacattctgcaattgatgtcaggtctcggggggaggatgcgaccagtcttcaacgtgaccatttccaacgtgcccggcccggaaggcacgctgtattatgaaggagcccggcttgaggccatgtatccggtatcgctaatcgctcacggcggcgccctgaacatcacctgcctgagctatgccggatcgctgaatttcggttttaccggctgtcgggatacgctgccgagcatgcagaaactggcggtttataccggtgaagctctggatgagctggaatcgctgattctgccacccaagaagcgcgcccgaacccgcaagtaa

SEQ ID NO: 2 - protein, 473 aa, Marinobacter hydrocarbonoclasticus
MKRLGTLDASWLAVESEDTPMHVGTLQIFSLPEGAPETFLRDMVTRMKEAGDVAPPWGYKLAWSGFLGRVIAPAWKVDKDIDLDYHVRHSALPRPGGERELGILVSRLHSNPLDFSRPLWECHVIEGLENNRFALYTKMHHSMIDGISGVRLMQRVLTTDPERCNMPPPWTVRPHQRRGAKTDKEASVPAAVSQAMDALKLQADMAPRLWQAGNRLVHSVRHPEDGLTAPFTGPVSVLNHRVTAQRRFATQHYQLDRLKNLAHASGGSLNDIVLYLCGTALRRFLAEQNNLPDTPLTAGIPVNIRPADDEGTGTQISFMIASLATDEADPLNRLQQIKTSTRRAKEHLQKLPKSALTQYTMLLMSPYILQLMSGLGGRMRPVFNVTISNVPGPEGTLYYEGARLEAMYPVSLIAHGGALNITCLSYAGSLNFGFTGCRDTLPSMQKLAVYTGEALDELESLILPPKKRARTRK

SEQ ID NO: 3 - DNA, 7314 bp, Artificial Sequence
  source: /note=“Description of Artificial Sequence Synthetic polynucleotide”
cactataccaattgagatgggctagtcaatgataattactagtccttttcctttgagttgtgggtatctgtaaattctgctagacctttgctggaaaacttgtaaattctgctagaccctctgtaaattccgctagacctttgtgtgttttttttgtttatattcaagtggttataatttatagaataaagaaagaataaaaaaagataaaaagaatagatcccagccctgtgtataactcactactttagtcagttccgcagtattacaaaaggatgtcgcaaacgctgtttgctcctctacaaaacagaccttaaaaccctaaaggcgtcggcatccgcttacagacaagctgtgaccgtctccgggagctgcatgtgtcagaggttttcaccgtcatcaccgaaacgcgcgaggcagcagatcaattcgcgcgcgaaggcgaagcggcatgcatttacgttgacaccatcgaatggtgcaaaacctttcgcggtatggcatgatagcgcccggaagagagtcaattcagggtggtgaatgtgaaaccagtaacgttatacgatgtcgcagagtatgccggtgtctcttatcagaccgtttcccgcgtggtgaaccaggccagccacgtttctgcgaaaacgcgggaaaaagtggaagcggcgatggcggagctgaattacattcccaaccgcgtggcacaacaactggcgggcaaacagtcgttgctgattggcgttgccacctccagtctggccctgcacgcgccgtcgcaaattgtcgcggcgattaaatctcgcgccgatcaactgggtgccagcgtggtggtgtcgatggtagaacgaagcggcgtcgaagcctgtaaagcggcggtgcacaatcttctcgcgcaacgcgtcagtgggctgatcattaactatccgctggatgaccaggatgccattgctgtggaagctgcctgcactaatgttccggcgttatttcttgatgtctctgaccagacacccatcaacagtattattttctcccatgaagacggtacgcgactgggcgtggagcatctggtcgcattgggtcaccagcaaatcgcgctgttagcgggcccattaagttctgtctcggcgcgtctgcgtctggctggctggcataaatatctcactcgcaatcaaattcagccgatagcggaacgggaaggcgactggagtgccatgtccggttttcaacaaaccatgcaaatgctgaatgagggcatcgttcccactgcgatgctggttgccaacgatcagatggcgctgggcgcaatgcgcgccattaccgagtccgggctgcgcgttggtgcggatatctcggtagtgggatacgacgataccgaagacagctcatgttatatcccgccgttaaccaccatcaaacaggattttcgcctgctggggcaaaccagcgtggaccgcttgctgcaactctctcagggccaggcggtgaagggcaatcagctgttgcccgtctcactggtgaaaagaaaaaccaccctggcgcccaatacgcaaaccgcctctccccgcgcgttggccgattcattaatgcagctggcacgacaggtttcccgactggaaagcgggcagtgagcgcaacgcaattaatgtaagttagcgcgaattgatctggtttgacagcttatcatcgactgcacggtgcaccaatgcttctggcgtcaggcagccatcggaagctgtggtatggctgtgcaggtcgtaaatcactgcataattcgtgtcgctcaaggcgcactcccgttctggataatgttttttgcgccgacatcataacggttctggcaaatattctgaaatgagctgttgacaattaatcatccggctcgtataatgtgtggaattgtgagcggataacaatttcacacaggaaacagcgccgctgagaaaaagcgaagcggcactgctctttaacaatttatcagacaatctgtgtgggcactcgaccggaattatcgattaactttattattaaaaattaaagaggtatatattaatgtatcgattaaataaggaggaataaaccatgaaacgtctcggaaccctggacgcctcctggctggcggttgaatctgaagacaccccgatgcatgtgggtacgcttcagattttctcactgccggaaggcgcaccagaaaccttcctgcgtgacatggtcactcgaatgaaagaggccggcgatgtggcaccaccctggggatacaaactggcctggtctggtttcctcgggcgcgtgatcgccccggcctggaaagtcgataaggatatcgatctggattatcacgtccggcactcagccctgcctcgccccggcggggagcgcgaactgggtattctggtatcccgactgcactctaaccccctggatttttcccgccctctttgggaatgccacgttattgaaggcctggagaataaccgttttgccctttacaccaaaatgcaccactcgatgattgacggcatcagcggcgtgcgactgatgcagagggtgctcaccaccgatcccgaacgctgcaatatgccaccgccctggacggtacgcccacaccaacgccgtggtgcaaaaaccgacaaagaggccagcgtgcccgcagcggtttcccaggcaatggacgccctgaagctccaggcagacatggcccccaggctgtggcaggccggcaatcgcctggtgcattcggttcgacacccggaagacggactgaccgcgcccttcactggaccggtttcggtgctcaatcaccgggttaccgcgcagcgacgttttgccacccagcattatcaactggaccggctgaaaaacctggcccatgcttccggcggttccttgaacgacatcgtgctttacctgtgtggcaccgcattgcggcgctttctggctgagcagaacaatctgccagacaccccgctgacggctggtataccggtgaatatccggccggcagacgacgagggtacgggcacccagatcagttttatgattgcctcgctggccaccgacgaagctgatccgttgaaccgcctgcaacagatcaaaacctcgacccgacgggccaaggagcacctgcagaaacttccaaaaagtgccctgacccagtacaccatgctgctgatgtcaccctacattctgcaattgatgtcaggtctcggggggaggatgcgaccagtcttcaacgtgaccatttccaacgtgcccggcccggaaggcacgctgtattatgaaggagcccggcttgaggccatgtatccggtatcgctaatcgctcacggcggcgccctgaacatcacctgcctgagctatgccggatcgctgaatttcggttttaccggctgtcgggatacgctgccgagcatgcagaaactggcggtttataccggtgaagctctggatgagctggaatcgctgattctgccacccaagaagcgcgcccgaacccgcaagtaactcgagatctgcagctggtaccatatgggaattcgaagcttgggcccgaacaaaaactcatctcagaagaggatctgaatagcgccgtcgaccatcatcatcatcatcattgagtttaaacggtctccagcttggctgttttggcggatgagagaagattttcagcctgatacagattaaatcagaacgcagaagcggtctgataaaacagaatttgcctggcggcagtagcgcggtggtcccacctgaccccatgccgaactcagaagtgaaacgccgtagcgccgatggtagtgtggggtctccccatgcgagagtagggaactgccaggcatcaaataaaacgaaaggctcagtcgaaagactgggcctttcgttttatctgttgtttgtcggtgaacgctctcctgacgcctgatgcggtattttctccttacgcatctgtgcggtatttcacaccgcatatggtgcactctcagtacaatctgctctgatgccgcatagttaagccagccccgacacccgccaacacccgctgacgagcttagtaaagccctcgctagattttaatgcggatgttgcgattacttcgccaactattgcgataacaagaaaaagccagcctttcatgatatatctcccaatttgtgtagggcttattatgcacgcttaaaaataataaaagcagacttgacctgatagtttggctgtgagcaattatgtgcttagtgcatctaacgcttgagttaagccgcgccgcgaagcggcgtcggcttgaacgaattgttagacattatttgccgactaccttggtgatctcgcctttcacgtagtggacaaattcttccaactgatctgcgcgcgaggccaagcgatcttcttcttgtccaagataagcctgtctagcttcaagtatgacgggctgatactgggccggcaggcgctccattgcccagtcggcagcgacatccttcggcgcgattttgccggttactgcgctgtaccaaatgcgggacaacgtaagcactacatttcgctcatcgccagcccagtcgggcggcgagttccatagcgttaaggtttcatttagcgcctcaaatagatcctgttcaggaaccggatcaaagagttcctccgccgctggacctaccaaggcaacgctatgttctcttgcttttgtcagcaagatagccagatcaatgtcgatcgtggctggctcgaagatacctgcaagaatgtcattgcgctgccattctccaaattgcagttcgcgcttagctggataacgccacggaatgatgtcgtcgtgcacaacaatggtgacttctacagcgcggagaatctcgctctctccaggggaagccgaagtttccaaaaggtcgttgatcaaagctcgccgcgttgtttcatcaagccttacggtcaccgtaaccagcaaatcaatatcactgtgtggcttcaggccgccatccactgcggagccgtacaaatgtacggccagcaacgtcggttcgagatggcgctcgatgacgccaactacctctgatagttgagtcgatacttcggcgatcaccgcttccctcatgatgtttaactttgttttagggcgactgccctgctgcgtaacatcgttgctgctccataacatcaaacatcgacccacggcgtaacgcgcttgctgcttggatgcccgaggcatagactgtaccccaaaaaaacagtcataacaagccatgaaaaccgccactgcgccgttaccaccgctgcgttcggtcaaggttctggaccagttgcgtgagcgcatacgctacttgcattacagcttacgaaccgaacaggcttatgtccactgggttcgtgccttcatccgtttccacggtgtgcgtcacccggcaaccttgggcagcagcgaagtcgaggcatttctgtcctggctggcgaacgagcgcaaggtttcggtctccacgcatcgtcaggcattggcggccttgctgttcttctacggcaaggtgctgtgcacggatctgccctggcttcaggagatcggaagacctcggccgtcgcggcgcttgccggtggtgctgaccccggatgaagtggttcgcatcctcggttttctggaaggcgagcatcgtttgttcgcccagcttctgtatggaacgggcatgcggatcagtgagggtttgcaactgcgggtcaaggatctggatttcgatcacggcacgatcatcgtgcgggagggcaagggctccaaggatcgggccttgatgttacccgagagcttggcacccagcctgcgcgagcaggggaattaattcccacgggttttgctgcccgcaaacgggctgttctggtgttgctagtttgttatcagaatcgcagatccggcttcagccggtttgccggctgaaagcgctatttcttccagaattgccatgattttttccccacgggaggcgtcactggctcccgtgttgtcggcagctttgattcgataagcagcatcgcctgtttcaggctgtctatgtgtgactgttgagctgtaacaagttgtctcaggtgttcaatttcatgttctagttgctttgttttactggtttcacctgttctattaggtgttacatgctgttcatctgttacattgtcgatctgttcatggtgaacagctttgaatgcaccaaaaactcgtaaaagctctgatgtatctatcttttttacaccgttttcatctgtgcatatggacagttttccctttgatatgtaacggtgaacagttgttctacttttgtttgttagtcttgatgcttcactgatagatacaagagccataagaacctcagatccttccgtatttagccagtatgttctctagtgtggttcgttgtttttgcgtgagccatgagaacgaaccattgagatcatacttactttgcatgtcactcaaaaattttgcctcaaaactggtgagctgaatttttgcagttaaagcatcgtgtagtgtttttcttagtccgttatgtaggtaggaatctgatgtaatggttgttggtattttgtcaccattcatttttatctggttgttctcaagttcggttacgagatccatttgtctatctagttcaacttggaaaatcaacgtatcagtcgggcggcctcgcttatcaaccaccaatttcatattgctgtaagtgtttaaatctttacttattggtttcaaaacccattggttaagccttttaaactcatggtagttattttcaagcattaacatgaacttaaattcatcaaggctaatctctatatttgccttgtgagttttcttttgtgttagttcttttaataaccactcataaatcctcatagagtatttgttttcaaaagacttaacatgttccagattatattttatgaatttttttaactggaaaagataaggcaatatctcttcactaaaaactaattctaatttttcgcttgagaacttggcatagtttgtccactggaaaatctcaaagcctttaaccaaaggattcctgatttccacagttctcgtcatcagctctctggttgctttagctaatacaccataagcattttccctactgatgttcatcatctgagcgtattggttataagtgaacgataccgtccgttctttccttgtagggttttcaatcgtggggttgagtagtgccacacagcataaaattagcttggtttcatgctccgttaagtcatagcgactaatcgctagttcatttgctttgaaaacaactaattcagacatacatctcaattggtctaggtgattttaat

SEQ ID NO: 4 - DNA, 30 bp, Artificial Sequence
  source: /note=“Description of Artificial Sequence Synthetic primer”
cccagatcagttttatgattgcctcgctgg

SEQ ID NO: 5 - DNA, 20 bp, Artificial Sequence
  source: /note=“Description of Artificial Sequence Synthetic primer”
atcatgaaacgtctcggaac

SEQ ID NO: 6 - DNA, 28 bp, Artificial Sequence
  source: /note=“Description of Artificial Sequence Synthetic primer”
cctcgagttacttgcgggttcgggcgcg

SEQ ID NO: 7 - DNA, 5199 bp, Artificial Sequence
  source: /note=“Description of Artificial Sequence Synthetic polynucleotide”
cactataccaattgagatgggctagtcaatgataattactagtccttttcctttgagttgtgggtatctgtaaattctgctagacctttgctggaaaacttgtaaattctgctagaccctctgtaaattccgctagacctttgtgtgttttttttgtttatattcaagtggttataatttatagaataaagaaagaataaaaaaagataaaaagaatagatcccagccctgtgtataactcactactttagtcagttccgcagtattacaaaaggatgtcgcaaacgctgtttcagtacactctctcaatacgaataaacggctcagaaatgagccgtttattttttctacccatatccttgaagcggtgttataatgccgcgccctcgatatggggatttttaacgacctgattttcgggtctcagtagtagttgacattagcggagcactaaaccatgaaacgtctcggaaccctggacgcctcctggctggcggttgaatctgaagacaccccgatgcatgtgggtacgcttcagattttctcactgccggaaggcgcaccagaaaccttcctgcgtgacatggtcactcgaatgaaagaggccggcgatgtggcaccaccctggggatacaaactggcctggtctggtttcctcgggcgcgtgatcgccccggcctggaaagtcgataaggatatcgatctggattatcacgtccggcactcagccctgcctcgccccggcggggagcgcgaactgggtattctggtatcccgactgcactctaaccccctggatttttcccgccctctttgggaatgccacgttattgaaggcctggagaataaccgttttgccctttacaccaaaatgcaccactcgatgattgacggcatcagcggcgtgcgactgatgcagagggtgctcaccaccgatcccgaacgctgcaatatgccaccgccctggacggtacgcccacaccaacgccgtggtgcaaaaaccgacaaagaggccagcgtgcccgcagcggtttcccaggcaatggacgccctgaagctccaggcagacatggcccccaggctgtggcaggccggcaatcgcctggtgcattcggttcgacacccggaagacggactgaccgcgcccttcactggaccggtttcggtgctcaatcaccgggttaccgcgcagcgacgttttgccacccagcattatcaactggaccggctgaaaaacctggcccatgcttccggcggttccttgaacgacatcgtgctttacctgtgtggcaccgcattgcggcgctttctggctgagcagaacaatctgccagacaccccgctgacggctggtataccggtgaatatccggccggcagacgacgagggtacgggcacccagatcagttttatgattgcctcgctggccaccgacgaagctgatccgttgaaccgcctgcaacagatcaaaacctcgacccgacgggccaaggagcacctgcagaaacttccaaaaagtgccctgacccagtacaccatgctgctgatgtcaccctacattctgcaattgatgtcaggtctcggggggaggatgcgaccagtcttcaacgtgaccatttccaacgtgcccggcccggaaggcacgctgtattatgaaggagcccggcttgaggccatgtatccggtatcgctaatcgctcacggcggcgccctgaacatcacctgcctgagctatgccggatcgctgaatttcggttttaccggctgtcgggatacgctgccgagcatgcagaaactggcggtttataccggtgaagctctggatgagctggaatcgctgattctgccacccaagaagcgcgcccgaacccgcaagtaactcgagatctgcagctggtaccatatgggaattcacccgctgacgagcttagtaaagccctcgctagattttaatgcggatgttgcgattacttcgccaactattgcgataacaagaaaaagccagcctttcatgatatatctcccaatttgtgtagggcttattatgcacgcttaaaaataataaaagcagacttgacctgatagtttggctgtgagcaattatgtgcttagtgcatctaacgcttgagttaagccgcgccgcgaagcggcgtcggcttgaacgaattgttagacattatttgccgactaccttggtgatctcgcctttcacgtagtggacaaattcttccaactgatctgcgcgcgaggccaagcgatcttcttcttgtccaagataagcctgtctagcttcaagtatgacgggctgatactgggccggcaggcgctccattgcccagtcggcagcgacatccttcggcgcgattttgccggttactgcgctgtaccaaatgcgggacaacgtaagcactacatttcgctcatcgccagcccagtcgggcggcgagttccatagcgttaaggtttcatttagcgcctcaaatagatcctgttcaggaaccggatcaaagagttcctccgccgctggacctaccaaggcaacgctatgttctcttgcttttgtcagcaagatagccagatcaatgtcgatcgtggctggctcgaagatacctgcaagaatgtcattgcgctgccattctccaaattgcagttcgcgcttagctggataacgccacggaatgatgtcgtcgtgcacaacaatggtgacttctacagcgcggagaatctcgctctctccaggggaagccgaagtttccaaaaggtcgttgatcaaagctcgccgcgttgtttcatcaagccttacggtcaccgtaaccagcaaatcaatatcactgtgtggcttcaggccgccatccactgcggagccgtacaaatgtacggccagcaacgtcggttcgagatggcgctcgatgacgccaactacctctgatagttgagtcgatacttcggcgatcaccgcttccctcatgatgtttaactttgttttagggcgactgccctgctgcgtaacatcgttgctgctccataacatcaaacatcgacccacggcgtaacgcgcttgctgcttggatgcccgaggcatagactgtaccccaaaaaaacagtcataacaagccatgaaaaccgccactgcgccgttaccaccgctgcgttcggtcaaggttctggaccagttgcgtgagcgcatacgctacttgcattacagcttacgaaccgaacaggcttatgtccactgggttcgtgccttcatccgtttccacggtgtgcgtcacccggcaaccttgggcagcagcgaagtcgaggcatttctgtcctggctggcgaacgagcgcaaggtttcggtctccacgcatcgtcaggcattggcggccttgctgttcttctacggcaaggtgctgtgcacggatctgccctggcttcaggagatcggaagacctcggccgtcgcggcgcttgccggtggtgctgaccccggatgaagtggttcgcatcctcggttttctggaaggcgagcatcgtttgttcgcccagcttctgtatggaacgggcatgcggatcagtgagggtttgcaactgcgggtcaaggatctggatttcgatcacggcacgatcatcgtgcgggagggcaagggctccaaggatcgggccttgatgttacccgagagcttggcacccagcctgcgcgagcaggggaattaattcccacgggttttgctgcccgcaaacgggctgttctggtgttgctagtttgttatcagaatcgcagatccggcttcagccggtttgccggctgaaagcgctatttcttccagaattgccatgattttttccccacgggaggcgtcactggctcccgtgttgtcggcagctttgattcgataagcagcatcgcctgtttcaggctgtctatgtgtgactgttgagctgtaacaagttgtctcaggtgttcaatttcatgttctagttgctttgttttactggtttcacctgttctattaggtgttacatgctgttcatctgttacattgtcgatctgttcatggtgaacagctttgaatgcaccaaaaactcgtaaaagctctgatgtatctatcttttttacaccgttttcatctgtgcatatggacagttttccctttgatatgtaacggtgaacagttgttctacttttgtttgttagtcttgatgcttcactgatagatacaagagccataagaacctcagatccttccgtatttagccagtatgttctctagtgtggttcgttgtttttgcgtgagccatgagaacgaaccattgagatcatacttactttgcatgtcactcaaaaattttgcctcaaaactggtgagctgaatttttgcagttaaagcatcgtgtagtgtttttcttagtccgttatgtaggtaggaatctgatgtaatggttgttggtattttgtcaccattcatttttatctggttgttctcaagttcggttacgagatccatttgtctatctagttcaacttggaaaatcaacgtatcagtcgggcggcctcgcttatcaaccaccaatttcatattgctgtaagtgtttaaatctttacttattggtttcaaaacccattggttaagccttttaaactcatggtagttattttcaagcattaacatgaacttaaattcatcaaggctaatctctatatttgccttgtgagttttcttttgtgttagttcttttaataaccactcataaatcctcatagagtatttgttttcaaaagacttaacatgttccagattatattttatgaatttttttaactggaaaagataaggcaatatctcttcactaaaaactaattctaatttttcgcttgagaacttggcatagtttgtccactggaaaatctcaaagcctttaaccaaaggattcctgatttccacagttctcgtcatcagctctctggttgctttagctaatacaccataagcattttccctactgatgttcatcatctgagcgtattggttataagtgaacgataccgtccgttctttccttgtagggttttcaatcgtggggttgagtagtgccacacagcataaaattagcttggtttcatgctccgttaagtcatagcgactaatcgctagttcatttgctttgaaaacaactaattcagacatacatctcaattggtctaggtgattttaat

SEQ ID NO: 8 - DNA, 169 bp, Escherichia coli
gctgtttcagtacactctctcaatacgaataaacggctcagaaatgagccgtttattttttctacccatatccttgaagcggtgttataatgccgcgccctcgatatggggatttttaacgacctgattttcgggtctcagtagtagttgacattagcggagcactaaa

SEQ ID NO: 9 - DNA, 42 bp, Artificial Sequence
  source: /note=“Description of Artificial Sequence Synthetic oligonucleotide”
aaaggatgtcgcaaacgctgtttcagtacactctctcaatac

SEQ ID NO: 10 - DNA, 34 bp, Artificial Sequence
  source: /note=“Description of Artificial Sequence Synthetic oligonucleotide”
gagctcggatccatggtttagtgctccgctaatg

SEQ ID NO: 11 - DNA, 5903 bp, Artificial Sequence
  source: /note=“Description of Artificial Sequence Synthetic polynucleotide”
cactataccaattgagatgggctagtcaatgataattactagtccttttcctttgagttgtgggtatctgtaaattctgctagacctttgctggaaaacttgtaaattctgctagaccctctgtaaattccgctagacctttgtgtgttttttttgtttatattcaagtggttataatttatagaataaagaaagaataaaaaaagataaaaagaatagatcccagccctgtgtataactcactactttagtcagttccgcagtattacaaaaggatgtcgcaaacgctgtttgctcctctacaaaacagaccttaaaaccctaaaggcgtcggcatccgcttacagacaagctgtgaccgtctccgggagctgcatgtgtcagaggttttcaccgtcatcaccgaaacgcgcgaggcagcagatcaattcgcgcgcgaaggcgaagcggcatgcatttacgttgacaccatcgaatggtgcaaaacctttcgcggtatggcatgatagcgcccggaagagagtcaattcagggtggtgaatgtgaaaccagtaacgttatacgatgtcgcagagtatgccggtgtctcttatcagaccgtttcccgcgtggtgaaccaggccagccacgtttctgcgaaaacgcgggaaaaagtggaagcggcgatggcggagctgaattacattcccaaccgcgtggcacaacaactggcgggcaaacagtcgttgctgattggcgttgccacctccagtctggccctgcacgcgccgtcgcaaattgtcgcggcgattaaatctcgcgccgatcaactgggtgccagcgtggtggtgtcgatggtagaacgaagcggcgtcgaagcctgtaaagcggcggtgcacaatcttctcgcgcaacgcgtcagtgggctgatcattaactatccgctggatgaccaggatgccattgctgtggaagctgcctgcactaatgttccggcgttatttcttgatgtctctgaccagacacccatcaacagtattattttctcccatgaagacggtacgcgactgggcgtggagcatctggtcgcattgggtcaccagcaaatcgcgctgttagcgggcccattaagttctgtctcggcgcgtctgcgtctggctggctggcataaatatctcactcgcaatcaaattcagccgatagcggaacgggaaggcgactggagtgccatgtccggttttcaacaaaccatgcaaatgctgaatgagggcatcgttcccactgcgatgctggttgccaacgatcagatggcgctgggcgcaatgcgcgccattaccgagtccgggctgcgcgttggtgcggatatctcggtagtgggatacgacgataccgaagacagctcatgttatatcccgccgttaaccaccatcaaacaggattttcgcctgctggggcaaaccagcgtggaccgcttgctgcaactctctcagggccaggcggtgaagggcaatcagctgttgcccgtctcactggtgaaaagaaaaaccaccctggcgcccaatacgcaaaccgcctctccccgcgcgttggccgattcattaatgcagctggcacgacaggtttcccgactggaaagcgggcagtgagcgcaacgcaattaatgtaagttagcgcgaattgatctggtttgacagcttatcatcgactgcacggtgcaccaatgcttctggcgtcaggcagccatcggaagctgtggtatggctgtgcaggtcgtaaatcactgcataattcgtgtcgctcaaggcgcactcccgttctggataatgttttttgcgccgacatcataacggttctggcaaatattctgaaatgagctgttgacaattaatcatccggctcgtataatgtgtggaattgtgagcggataacaatttcacacaggaaacagcgccgctgagaaaaagcgaagcggcactgctctttaacaatttatcagacaatctgtgtgggcactcgaccggaattatcgattaactttattattaaaaattaaagaggtatatattaatgtatcgattaaataaggaggaataaaccatggatccgagctcgagatctgcagctggtaccatatgggaattcgaagcttgggcccgaacaaaaactcatctcagaagaggatctgaatagcgccgtcgaccatcatcatcatcatcattgagtttaaacggtctccagcttggctgttttggcggatgagagaagattttcagcctgatacagattaaatcagaacgcagaagcggtctgataaaacagaatttgcctggcggcagtagcgcggtggtcccacctgaccccatgccgaactcagaagtgaaacgccgtagcgccgatggtagtgtggggtctccccatgcgagagtagggaactgccaggcatcaaataaaacgaaaggctcagtcgaaagactgggcctttcgttttatctgttgtttgtcggtgaacgctctcctgacgcctgatgcggtattttctccttacgcatctgtgcggtatttcacaccgcatatggtgcactctcagtacaatctgctctgatgccgcatagttaagccagccccgacacccgccaacacccgctgacgagcttagtaaagccctcgctagattttaatgcggatgttgcgattacttcgccaactattgcgataacaagaaaaagccagcctttcatgatatatctcccaatttgtgtagggcttattatgcacgcttaaaaataataaaagcagacttgacctgatagtttggctgtgagcaattatgtgcttagtgcatctaacgcttgagttaagccgcgccgcgaagcggcgtcggcttgaacgaattgttagacattatttgccgactaccttggtgatctcgcctttcacgtagtggacaaattcttccaactgatctgcgcgcgaggccaagcgatcttcttcttgtccaagataagcctgtctagcttcaagtatgacgggctgatactgggccggcaggcgctccattgcccagtcggcagcgacatccttcggcgcgattttgccggttactgcgctgtaccaaatgcgggacaacgtaagcactacatttcgctcatcgccagcccagtcgggcggcgagttccatagcgttaaggtttcatttagcgcctcaaatagatcctgttcaggaaccggatcaaagagttcctccgccgctggacctaccaaggcaacgctatgttctcttgcttttgtcagcaagatagccagatcaatgtcgatcgtggctggctcgaagatacctgcaagaatgtcattgcgctgccattctccaaattgcagttcgcgcttagctggataacgccacggaatgatgtcgtcgtgcacaacaatggtgacttctacagcgcggagaatctcgctctctccaggggaagccgaagtttccaaaaggtcgttgatcaaagctcgccgcgttgtttcatcaagccttacggtcaccgtaaccagcaaatcaatatcactgtgtggcttcaggccgccatccactgcggagccgtacaaatgtacggccagcaacgtcggttcgagatggcgctcgatgacgccaactacctctgatagttgagtcgatacttcggcgatcaccgcttccctcatgatgtttaactttgttttagggcgactgccctgctgcgtaacatcgttgctgctccataacatcaaacatcgacccacggcgtaacgcgcttgctgcttggatgcccgaggcatagactgtaccccaaaaaaacagtcataacaagccatgaaaaccgccactgcgccgttaccaccgctgcgttcggtcaaggttctggaccagttgcgtgagcgcatacgctacttgcattacagcttacgaaccgaacaggcttatgtccactgggttcgtgccttcatccgtttccacggtgtgcgtcacccggcaaccttgggcagcagcgaagtcgaggcatttctgtcctggctggcgaacgagcgcaaggtttcggtctccacgcatcgtcaggcattggcggccttgctgttcttctacggcaaggtgctgtgcacggatctgccctggcttcaggagatcggaagacctcggccgtcgcggcgcttgccggtggtgctgaccccggatgaagtggttcgcatcctcggttttctggaaggcgagcatcgtttgttcgcccagcttctgtatggaacgggcatgcggatcagtgagggtttgcaactgcgggtcaaggatctggatttcgatcacggcacgatcatcgtgcgggagggcaagggctccaaggatcgggccttgatgttacccgagagcttggcacccagcctgcgcgagcaggggaattaattcccacgggttttgctgcccgcaaacgggctgttctggtgttgctagtttgttatcagaatcgcagatccggcttcagccggtttgccggctgaaagcgctatttcttccagaattgccatgattttttccccacgggaggcgtcactggctcccgtgttgtcggcagctttgattcgataagcagcatcgcctgtttcaggctgtctatgtgtgactgttgagctgtaacaagttgtctcaggtgttcaatttcatgttctagttgctttgttttactggtttcacctgttctattaggtgttacatgctgttcatctgttacattgtcgatctgttcatggtgaacagctttgaatgcaccaaaaactcgtaaaagctctgatgtatctatcttttttacaccgttttcatctgtgcatatggacagttttccctttgatatgtaacggtgaacagttgttctacttttgtttgttagtcttgatgcttcactgatagatacaagagccataagaacctcagatccttccgtatttagccagtatgttctctagtgtggttcgttgtttttgcgtgagccatgagaacgaaccattgagatcatacttactttgcatgtcactcaaaaattttgcctcaaaactggtgagctgaatttttgcagttaaagcatcgtgtagtgtttttcttagtccgttatgtaggtaggaatctgatgtaatggttgttggtattttgtcaccattcatttttatctggttgttctcaagttcggttacgagatccatttgtctatctagttcaacttggaaaatcaacgtatcagtcgggcggcctcgcttatcaaccaccaatttcatattgctgtaagtgtttaaatctttacttattggtttcaaaacccattggttaagccttttaaactcatggtagttattttcaagcattaacatgaacttaaattcatcaaggctaatctctatatttgccttgtgagttttcttttgtgttagttcttttaataaccactcataaatcctcatagagtatttgttttcaaaagacttaacatgttccagattatattttatgaatttttttaactggaaaagataaggcaatatctcttcactaaaaactaattctaatttttcgcttgagaacttggcatagtttgtccactggaaaatctcaaagcctttaaccaaaggattcctgatttccacagttctcgtcatcagctctctggttgctttagctaatacaccataagcattttccctactgatgttcatcatctgagcgtattggttataagtgaacgataccgtccgttctttccttgtagggttttcaatcgtggggttgagtagtgccacacagcataaaattagcttggtttcatgctccgttaagtcatagcgactaatcgctagttcatttgctttgaaaacaactaattcagacatacatctcaattggtctaggtgattttaat

SEQ ID NO: 12 - DNA, 27 bp, Artificial Sequence
  source: /note=“Description of Artificial Sequence Synthetic oligonucleotide”
atatgacgtcggcatccgcttacagac

SEQ ID NO: 13 - DNA, 32 bp, Artificial Sequence
  source: /note=“Description of Artificial Sequence Synthetic oligonucleotide”
aattcttaagtcaggagagcgttcaccgacaa

SEQ ID NO: 14 - DNA, 24 bp, Artificial Sequence
  source: /note=“Description of Artificial Sequence Synthetic oligonucleotide”
gaattccacccgctgacgagctta

SEQ ID NO: 15 - DNA, 21 bp, Artificial Sequence
  source: /note=“Description of Artificial Sequence Synthetic oligonucleotide”
cgaattcccatatggtaccag

SEQ ID NO: 16 - DNA, 1368 bp, Marinobacter hydrocarbonoclasticus
atgacgcccctgaatcccactgaccagctctttctctggctggaaaaacgccagcagcccatgcatgtgggcggcctccagctgttttccttccccgaaggcgcgccggacgactatgtcgcgcagctggcagaccagcttcggcagaagacggaggtgaccgccccctttaaccagcgcctgagctatcgcctgggccagccggtatgggtggaggatgagcacctggaccttgagcatcatttccgcttcgaggcgctgcccacacccgggcgtattcgggagctgctgtcgttcgtatcggcggagcattcgcacctgatggaccgggagcgccccatgtgggaggtgcacctgatcgagggcctgaaagaccggcagtttgcgctctacaccaaggttcaccattccctggtggacggtgtctcggccatgcgcatggccacccggatgctgagtgaaaacccggacgaacacggcatgccgccaatctgggatctgccttgcctgtcacgggataggggtgagtcggacggacactccctctggcgcagtgtcacccatttgctggggctttcggaccgccagctcggcaccattcccactgtggcaaaggagctactgaaaaccatcaatcaggcccggaaggatccggcctacgactccattttccatgccccgcgctgcatgctgaaccagaaaatcaccggttcccgtcgattcgccgctcagtcctggtgcctgaaacggattcgcgccgtatgcgaggcctacggcaccacggtcaacgatgtcgtgactgccatgtgcgcagcggctctgcgtacctatctgatgaatcaggatgccttgccggagaaaccactggtggcctttgtgccggtgtcgctacgccgggacgacagctccggcggcaaccaggtaggcgtcatcctggcgagccttcacaccgatgtgcaggacgccggcgaacgactgttaaaaattcaccacggcatggaagaggccaagcagcgctaccggcatatgagcccggaggaaatcgtcaactacacggccctgaccctggcgccggccgccttccacctgctgaccgggctggcgcccaagtggcagaccttcaatgtggtgatttccaatgtccccgggccatccaggcccctgtactggaacggggcgaaactggaaggcatgtatccggtgtctatcgatatggacaggctggccctgaacatgacactgaccagctataacgaccaggtggagttcggcctgattggctgtcgccggaccctgcccagcctgcaacggatgctggactacctggaacagggtctggcagagctggagctcaacgccggtctgtaa

SEQ ID NO: 17 - protein, 455 aa, Marinobacter hydrocarbonoclasticus
MTPLNPTDQLFLWLEKRQQPMHVGGLQLFSFPEGAPDDYVAQLADQLRQKTEVTAPFNQRLSYRLGQPVWVEDEHLDLEHHFRFEALPTPGRIRELLSFVSAEHSHLMDRERPMWEVHLIEGLKDRQFALYTKVHHSLVDGVSAMRMATRMLSENPDEHGMPPIWDLPCLSRDRGESDGHSLWRSVTHLLGLSDRQLGTIPTVAKELLKTINQARKDPAYDSIFHAPRCMLNQKITGSRRFAAQSWCLKRIRAVCEAYGTTVNDVVTAMCAAALRTYLMNQDALPEKPLVAFVPVSLRRDDSSGGNQVGVILASLHTDVQDAGERLLKIHHGMEEAKQRYRHMSPEEIVNYTALTLAPAAFHLLTGLAPKWQTFNVVISNVPGPSRPLYWNGAKLEGMYPVSIDMDRLALNMTLTSYNDQVEFGLIGCRRTLPSLQRMLDYLEQGLAELELNAGL

SEQ ID NO: 18 - DNA, 1374 bp, Alcanivorax borkumensis
atgaaagcgcttagcccagtggatcaactgttcctgtggctggaaaaacgacagcaacccatgcacgtaggcggtttgcagctgttttccttcccggaaggtgccggccccaagtatgtgagtgagctggcccagcaaatgcgggattactgccacccagtggcgccattcaaccagcgcctgacccgtcgactcggccagtattactggactagagacaaacagttcgatatcgaccaccacttccgccacgaagcactccccaaacccggtcgcattcgcgaactgctttctttggtctccgccgaacattccaacctgctggaccgggagcgccccatgtgggaagcccatttgatcgaagggatccgcggtcgccagttcgctctctattataagatccaccattcggtgatggatggcatatccgccatgcgtatcgcctccaaaacgctttccactgaccccagtgaacgtgaaatggctccggcttgggcgttcaacaccaaaaaacgctcccgctcactgcccagcaacccggttgacatggcctccagcatggcgcgcctaaccgcgagcataagcaaacaagctgccacagtgcccggtctcgcgcgggaggtttacaaagtcacccaaaaagccaaaaaagatgaaaactatgtgtctatttttcaggctcccgacacgattctgaataataccatcaccggttcacgccgctttgccgcccagagctttccattaccgcgcctgaaagttatcgccaaggcctataactgcaccattaacaccgtggtgctctccatgtgtggccacgctctgcgcgaatacttgattagccaacacgcgctgcccgatgagccactgattgccatggtgcccatgagcctgcggcaggacgacagcactggcggcaaccagatcggtatgatcttggctaacctgggcacccacatctgtgatccagctaatcgcctgcgcgtcatccacgattccgtcgaggaagccaaatcccgcttctcgcagatgagcccggaagaaattctcaatttcaccgccctcaccatggctcccaccggcttgaacttactgaccggcctagcgccaaaatggcgggccttcaacgtggtgatttccaacatacccgggccgaaagagccgctgtactggaatggtgcacagctgcaaggagtgtatccagtatccattgccttggatcgcatcgccctaaatatcaccctcaccagttatgtagaccagatggaatttgggcttatcgcctgccgccgtactctgccttccatgcagcgactactggattacctggaacagtccatccgcgaattggaaatcggtgcaggaattaaatag

SEQ ID NO: 19 - protein, 457 aa, Alcanivorax borkumensis
MKALSPVDQLFLWLEKRQQPMHVGGLQLFSFPEGAGPKYVSELAQQMRDYCHPVAPFNQRLTRRLGQYYWTRDKQFDIDHHFRHEALPKPGRIRELLSLVSAEHSNLLDRERPMWEAHLIEGIRGRQFALYYKIHHSVMDGISAMRIASKTLSTDPSEREMAPAWAFNTKKRSRSLPSNPVDMASSMARLTASISKQAATVPGLAREVYKVTQKAKKDENYVSIFQAPDTILNNTITGSRRFAAQSFPLPRLKVIAKAYNCTINTVVLSMCGHALREYLISQHALPDEPLIAMVPMSLRQDDSTGGNQIGMILANLGTHICDPANRLRVIHDSVEEAKSRFSQMSPEEILNFTALTMAPTGLNLLTGLAPKWRAFNVVISNIPGPKEPLYWNGAQLQGVYPVSIALDRIALNITLTSYVDQMEFGLIACRRTLPSMQRLLDYLEQSIRELEIGAGIK

SEQ ID NO: 20 - DNA, 1356 bp, Alcanivorax borkumensis
atggcccgtaaattgtctattatggattccggctggttaatgatggagacccgggaaacccctatgcatgtgggggggttggcgttgtttgccattccagaaggtgctcctgaggattatgtggaaagtatctatcgatacctggtggatgtggatagcatctgccgcccatttaaccaaaagattcagtctcatttgcccctgtacttagatgctacttgggtggaagacaaaaatttcgatattgactaccacgtacggcattctgccttgcctcggccgggacgggtgcgtgagctgttggcgttagtatcgcggttgcacgcccagcgtttggatcctagccgcccgttgtgggagagctatttgatcgaggggttggagggaaaccgtttcgctctttataccaagatgcatcactccatggtggatggggtggcagggatgcacctaatgcagtctcgcctagctacttgtgcggaagaccgtttacccgccccttggtctggcgagtgggatgcagagaagaaaccgagaaagagccgtggcgctgcagcggcgaatgccggtatgaaaggaacaatgaataacctgcgccgaggtggtggtcagcttgtggacctgctgcgacagcccaaggatggcaacgtaaagactatctatcgggcgccgaaaacccagctaaaccgccgggtgacgggcgcgcgacgctttgctgcccagtcgtggtcgctgtcgcggattaaagccgcgggcaaacagcatggcggtacggtgaatgatattttccttgccatgtgtggcggcgcgctgcgtcgctatctgctcagtcaggatgccttgtccgatcagccgttggtagcccaggtgccagtagccttgcgtagtgcggatcaggctggtgagggtggcaatgccattactacggttcaggtaagcctgggtacgcatattgctcagccgctgaatcggctggccgcaatccaggattccatgaaagcggtgaaatctcggcttggtgatatgcagaagtccgagatcgatgtttatacggtgctgaccaatatgccgctgtctttggggcaggtcacgggcctgtccgggcgcgtaagccccatgtttaacctagtgatttccaatgtgccggggccgaaggaaacgcttcatctcaatggtgcggagatgttggctacctatccggtgtcattggttctgcatggttacgccctaaatatcactgtggtgagctacaagaatagccttgagtttggcgtgatcggttgccgtgacacgttgcctcatattcagcgttttctggtttatctcgaagaatcgctggtggagctggagccttga

SEQ ID NO: 21 - protein, 451 aa, Alcanivorax borkumensis
MARKLSIMDSGWLMMETRETPMHVGGLALFAIPEGAPEDYVESIYRYLVDVDSICRPFNQKIQSHLPLYLDATWVEDKNFDIDYHVRHSALPRPGRVRELLALVSRLHAQRLDPSRPLWESYLIEGLEGNRFALYTKMHHSMVDGVAGMHLMQSRLATCAEDRLPAPWSGEWDAEKKPRKSRGAAAANAGMKGTMNNLRRGGGQLVDLLRQPKDGNVKTIYRAPKTQLNRRVTGARRFAAQSWSLSRIKAAGKQHGGTVNDIFLAMCGGALRRYLLSQDALSDQPLVAQVPVALRSADQAGEGGNAITTVQVSLGTHIAQPLNRLAAIQDSMKAVKSRLGDMQKSEIDVYTVLTNMPLSLGQVTGLSGRVSPMFNLVISNVPGPKETLHLNGAEMLATYPVSLVLHGYALNITVVSYKNSLEFGVIGCRDTLPHIQRFLVYLEESLVELEP

(19) United States
(12) Patent Application Publication
(10) Pub. No.: US 2014/0215904 A1
(43) Pub. Date: Aug. 7, 2014

(54) COMPOSITIONS COMPRISING AND METHODS FOR PRODUCING BETA-HYDROXY FATTY ACID ESTERS

(71) Applicants: Archana Pandey, San Francisco, CA (US); Mathew A Rude, San Francisco, CA (US); Fernando A Sanchez-Riera, South San Francisco, CA (US); Bernardo M. da Costa, South San Francisco, CA (US)

(72) Inventors: Archana Pandey, San Francisco, CA (US); Mathew A Rude, San Francisco, CA (US); Fernando A Sanchez-Riera, South San Francisco, CA (US); Bernardo M. da Costa, South San Francisco, CA (US)

(73) Assignee: LS9, Inc., San Francisco, CA (US)

(21) Appl. No.: 14/007,829

(22) PCT Filed: Mar. 30, 2012
(86) PCT No.: PCT/US12/31682

§ 371 Date: Feb. 28, 2014

Related U.S. Application Data
(60) Provisional application No. 61/469,425, filed on Mar. 30, 2011.

(51) Int. Cl. C12P 7/64; C10L 1/02

(52) U.S. Cl. CPC C12P 7/649; C10L 1/02; C10L 1/19
USPC 44/400; 435/252.3; 435/134; 435/254.2; 435/252.33

(57) ABSTRACT

Disclosed are fatty ester compositions comprising beta-hydroxy fatty esters, as well as methods for producing beta-hydroxy fatty esters, and recombinant microorganisms useful in methods of producing beta-hydroxy fatty esters

### CROSS-REFERENCE TO RELATED APPLICATIONS

This application claims priority benefit to U.S. application Ser. No. 61/469,425, filed Mar. 30, 2011, which is expressly incorporated by reference herein in their entirety.

### INCORPORATION-BY-REFERENCE OF MATERIAL SUBMITTED ELECTRONICALLY

The instant application contains a Sequence Listing which has been submitted in ASCII format via EFS-Web and is hereby incorporated by reference in its entirety. Said ASCII copy, created on Mar. 30, 2012, is named LS035PCT.txt and is 50,970 bytes in size.

### BACKGROUND OF THE INVENTION

Crude petroleum is a very complex mixture containing a wide range of hydrocarbons. It is converted into a diversity of fuels and chemicals through a variety of chemical processes in refineries. Crude petroleum is a source of transportation fuels as well as a source of raw materials for producing petrochemicals. Petrochemicals are used to make specialty chemicals such as plastics, resins, fibers, elastomers, pharmaceuticals, lubricants, and gels.

The most important transportation fuels—gasoline, diesel, and jet fuel—contain distinctively different mixtures of hydrocarbons which are tailored toward optimal engine performance. For example, gasoline comprises straight chain, branched chain, and aromatic hydrocarbons generally ranging from about 4 to 12 carbon atoms, while diesel predominantly comprises straight chain hydrocarbons ranging from about 9 to 23 carbon atoms. Diesel fuel quality is evaluated by parameters such as cetane number, kinematic viscosity, oxidative stability, and cloud point (Knothe G., Fuel Process Technol. 86:1059-1070 (2005)). These parameters, among others, are impacted by the hydrocarbon chain length as well as by the degree of branching or saturation of the hydrocarbon.

Microbially-produced fatty acid derivatives can be tailored by genetic manipulation. Metabolic engineering enables microbial strains to produce various mixtures of fatty acid derivatives, which can be optimized, for example, to meet or exceed fuel standards or other commercially relevant product specifications. Microbial strains can be engineered to produce chemicals or precursor molecules that are typically derived from petroleum. In some instances, it is desirable to mimic the product profile of an existing product, for example the product profile of an existing petroleum-derived fuel or chemical product, for efficient drop-in compatibility or substitution. Recombinant cells and methods described herein demonstrate microbial production of fatty acid derivatives with varied ratios of odd: even length chains as a means to precisely control the structure and function of, e.g., hydrocarbon-based fuels and chemicals.

There is a need for cost-effective alternatives to petroleum products that do not require exploration, extraction, transportation over long distances, or substantial refinement, and avoid the types of environmental damage associated with processing of petroleum. For similar reasons, there is a need for alternative sources of chemicals which are typically derived from petroleum. There is also a need for efficient and cost-effective methods for producing high-quality biofuels, fuel alternatives, and chemicals from renewable energy sources.

Recombinant microbial cells engineered to produce fatty acid precursor molecules and fatty acid derivatives made therefrom, methods using these recombinant microbial cells to produce compositions comprising fatty acid derivatives having desired properties and compositions produced by these methods, address these needs.

### SUMMARY OF THE INVENTION

The invention provides novel host cells engineered to produce fatty ester compositions comprising beta hydroxy esters, as well as cell cultures which comprise such cells, methods of using such cells to make fatty ester compositions comprising beta hydroxy esters, fatty ester compositions comprising beta hydroxy esters, and other features apparent upon further review.

The recombinant microorganism comprises a heterologous polynucleotide sequence encoding a polypeptide having ester synthase activity (EC 2.3.1.75), wherein in the presence of a carbon source the recombinant microorganism produces an ester composition comprising beta-hydroxy esters.

The recombinant microorganism may further comprise a heterologous polynucleotide sequence encoding a thioesterase (EC 3.1.2.14 or EC 3.1.1.5) and/or an acyl-CoA synthase EC 2.3.1.86).

### BRIEF DESCRIPTION OF THE DRAWINGS

FIGS. 1A and B are GC-FID traces of a derivatized fatty acid ethyl ester (FAEE) (FIG. 1A) and a derivatized fatty acid methyl ester (FAME) (FIG. 1B). The sample (yellow trace) is overlaid with the standards (white trace). The overlay is done for the top chromatogram.

FIG. 2 is a GC-MS chromatogram of derivatized FAEE with peaks co eluting for regular FAEE and beta-hydroxy FAEE. A beta-hydroxy ester was identified for all the 4 compounds (C12, C14, C16 and C18 beta-hydroxy FAEE).

FIG. 3 is a GC-MS chromatogram of underivatized FAEE where C14:1 beta-hydroxy and C14:0 Beta-hydroxy elute separately on the chromatogram.

FIGS. 4A and B provide GC-MS chromatograms of derivatized FAME where beta-hydroxy FAME was identified for all the 4 compounds (C12, C14, C16 and C18 beta-hydroxy FAME; FIG. 4A). The mass spectra shown is of C16 beta-hydroxy FAME (FIG. 4B).

FIG. 5 presents an overview of two exemplary biosynthetic pathways for production of fatty esters starting with acyl-ACP, where the production of fatty esters is accomplished by a one enzyme system or a three enzyme system.

### DETAILED DESCRIPTION OF THE INVENTION

The invention is based, at least in part, on the production of fatty ester compositions by genetically engineered host cells, wherein the compositions comprises beta-hydroxy fatty esters. Examples of fatty esters include fatty acid esters, such as those derived from short-chain alcohols, including, for example, beta-hydroxy fatty acid methyl ester (“FAME”) and beta-hydroxy fatty acid ethyl ester (“FAEE”), and those derived from longer chain fatty alcohols. A fatty ester composition comprising beta-hydroxy fatty esters may be used, individually or in suitable combinations, as a biofuel (e.g., a biodiesel), an industrial chemical, or a component of, or feedstock for, a biofuel or an industrial chemical. In some aspects, the beta-hydroxy ester is separated from the fatty ester composition. In other aspects, the invention pertains to a method of producing one or more free fatty ester compositions comprising one or more fatty acid derivatives such as beta-hydroxy fatty acid esters, for example, FAME, FAEE and/or other fatty acid ester derivatives of longer-chain alcohols.

The inventors have engineered microorganisms to express an exogenous polynucleotide sequence encoding a polypeptide having ester synthase activity, which is effective to produce a fatty ester composition comprising a beta-hydroxy fatty ester, such as a beta-hydroxy fatty acid methyl ester or a beta-hydroxy fatty acid ethyl ester, when cultured in the presence of a carbon source and an alcohol.

Production of fatty acid esters by recombinant microorganisms has been described for example in PCT Publication Nos. WO07/136,762, WO08/119,082, WO2010/022090, WO2010/118409, WO/2011/127409 and WO/2011/038132, each of which is expressly incorporated by reference herein. The invention is intended to encompass the use of any suitable ester synthase, which includes any polypeptide that, when expressed in a microorganism in the presence of a carbon source and an alcohol, catalyzes the production of fatty esters, e.g., fatty acid methyl and ethyl esters, including beta-hydroxy esters. The ester synthase may utilize one or both of acyl-ACP and acyl-CoA as a substrate to generate fatty acid methyl and ethyl esters.

As one of ordinary skill in the art will appreciate, the methods of the invention can be practiced using fermentation processes described herein or using any suitable fermentation conditions or methods, including those known to those of ordinary skill in the art. For example, it is envisioned that the fermentation processes can be scaled up using the methods described herein or alternative methods known in the art. It is further envisioned that any suitable carbon source may be used, including, for example, biomass of any source.

### DEFINITIONS

As used in this specification and the appended claims, the singular forms “a,” “an” and “the” include plural referents unless the context clearly dictates otherwise. Thus, for example, reference to “a recombinant host cell” includes two or more such recombinant host cells, reference to “a fatty ester” includes one or more fatty esters, or mixtures of fatty esters, reference to “a nucleic acid coding sequence” includes one or more nucleic acid coding sequences, reference to “an enzyme” includes one or more enzymes, and the like.

Unless defined otherwise, all technical and scientific terms used herein have the same meaning as commonly understood by one of ordinary skill in the art to which the invention pertains. Although other methods and materials similar, or equivalent, to those described herein can be used in the practice of the present invention, the preferred materials and methods are described herein.

In describing and claiming the present invention, the following terminology will be used in accordance with the definitions set out below.

Accession Numbers: Sequence Accession numbers throughout this description were obtained from databases provided by the NCBI (National Center for Biotechnology Information) maintained by the National Institutes of Health, U.S.A. (which are identified herein as “NCBI Accession Numbers” or alternatively as “GenBank Accession Numbers”), and from the UniProt Knowledgebase (UniProtKB) and Swiss-Prot databases provided by the Swiss Institute of Bioinformatics (which are identified herein as “UniProtKB Accession Numbers”).

Enzyme Classification (EC) Numbers: EC numbers are established by the Nomenclature Committee of the International Union of Biochemistry and Molecular Biology (IUBMB), description of which is available on the IUBMB Enzyme Nomenclature website on the World Wide Web. EC numbers classify enzymes according to the reaction catalyzed.

As used herein, the term “nucleotide” refers to a monomeric unit of a polynucleotide that consists of a heterocyclic base, a sugar, and one or more phosphate groups. The naturally occurring bases (guanine, (G), adenine, (A), cytosine, (C), thymine, (T), and uracil (U)) are typically derivatives of purine or pyrimidine, though it should be understood that naturally and non-naturally occurring base analogs are also included. The naturally occurring sugar is the pentose (five-carbon sugar) deoxyribose (which forms DNA) or ribose (which forms RNA), though it should be understood that naturally and non-naturally occurring sugar analogs are also included. Nucleic acids are typically linked via phosphate bonds to form nucleic acids or polynucleotides, though many other linkages are known in the art (e.g., phosphorothioates, boranophosphates, and the like).

As used herein, the term “polynucleotide” refers to a polymer of ribonucleotides (RNA) or deoxyribonucleotides (DNA), which can be single-stranded or double-stranded and which can contain non-natural or altered nucleotides. The terms “polynucleotide,” “nucleic acid sequence,” and “nucleotide sequence” are used interchangeably herein to refer to a polymeric form of nucleotides of any length, either RNA or DNA. These terms refer to the primary structure of the molecule, and thus include double- and single-stranded DNA, and double- and single-stranded RNA. The terms include, as equivalents, analogs of either RNA or DNA made from nucleotide analogs and modified polynucleotides such as, though not limited to methylated and/or capped polynucleotides. The polynucleotide can be in any form, including but not limited to, plasmid, viral, chromosomal, EST, cDNA, mRNA, and rRNA.

As used herein, the terms “polypeptide” and “protein” are used interchangeably to refer to a polymer of amino acid residues. The term “recombinant polypeptide” refers to a polypeptide that is produced by recombinant techniques, wherein generally DNA or RNA encoding the expressed protein is inserted into a suitable expression vector that is in turn used to transform a host cell to produce the polypeptide.

As used herein, the terms “homolog,” and “homologous” refer to a polynucleotide or a polypeptide comprising a sequence that is at least about 50% identical to the corresponding polynucleotide or polypeptide sequence. Preferably homologous polynucleotides or polypeptides have polynucleotide sequences or amino acid sequences that have at least about 80%, 81%, 82%, 83%, 84%, 85%, 86%, 87%, 88%, 89%, 90%, 91%, 2%, 93%, 94%, 95%, 96%, 97%, 98% or at least about 99% homology to the corresponding amino acid sequence or polynucleotide sequence. As used herein the terms sequence “homology” and sequence “identity” are used interchangeably.

One of ordinary skill in the art is well aware of methods to determine homology between two or more sequences. Briefly, calculations of “homology” between two sequences can be performed as follows. The sequences are aligned for optimal comparison purposes (e.g., gaps can be introduced in one or both of a first and a second amino acid or nucleic acid sequence for optimal alignment and non-homologous sequences can be disregarded for comparison purposes).

In a preferred embodiment, the length of a first sequence that is aligned for comparison purposes is at least about 30%, preferably at least about 40%, more preferably at least about 50%, even more preferably at least about 60%, and even more preferably at least about 70%, at least about 80%, at least about 90%, or about 100% of the length of a second sequence. The amino acid residues or nucleotides at corresponding amino acid positions or nucleotide positions of the first and second sequences are then compared. When a position in the first sequence is occupied by the same amino acid residue or nucleotide as the corresponding position in the second sequence, then the molecules are identical at that position. The percent homology between the two sequences is a function of the number of identical positions shared by the sequences, taking into account the number of gaps and the length of each gap, that need to be introduced for optimal alignment of the two sequences.

The comparison of sequences and determination of percent homology between two sequences can be accomplished using a mathematical algorithm, such as BLAST (Altschul et al., J. Mol. Biol., 215(3): 403-410 (1990)). The percent homology between two amino acid sequences also can be determined using the Needleman and Wunsch algorithm that has been incorporated into the GAP program in the GCG software package, using either a Blossum 62 matrix or a PAM250 matrix, and a gap weight of 16, 14, 12, 10, 8, 6, or 4 and a length weight of 1, 2, 3, 4, 5, or 6 (Needleman and Wunsch, J. Mol. Biol., 48: 444-453 (1970)). The percent homology between two nucleotide sequences also can be determined using the GAP program in the GCG software package, using a NWSgapdna.CMP matrix and a gap weight of 40, 50, 60, 70, or 80 and a length weight of 1, 2, 3, 4, 5, or 6. One of ordinary skill in the art can perform initial homology calculations and adjust the algorithm parameters accordingly. A preferred set of parameters (and the one that should be used if a practitioner is uncertain about which parameters should be applied to determine if a molecule is within a homology limitation of the claims) are a Blossum 62 scoring matrix with a gap penalty of 12, a gap extend penalty of 4, and a frameshift gap penalty of 5. Additional methods of sequence alignment are known in the biotechnology arts (see, e.g., Rosenberg, BMC Bioinformatics, 6: 278 (2005); Altschul, et al., FEBS J., 272(20): 5101-5109 (2005)).

As used herein, the term “hybridizes under low stringency, medium stringency, high stringency, or very high stringency conditions” describes conditions for hybridization and washing. Guidance for performing hybridization reactions can be found in Current Protocols in Molecular Biology, John Wiley & Sons, N.Y. (1989), 6.3.1-6.3.6. Aqueous and non-aqueous methods are described in that reference and either method can be used. Specific hybridization conditions referred to herein are as follows: 1) low stringency hybridization conditions—6× sodium chloride/sodium citrate (SSC) at about 45° C., followed by two washes in 0.2×SSC, 0.1% SDS at least at 50° C. (the temperature of the washes can be increased to 55° C. for low stringency conditions); 2) medium stringency hybridization conditions—6×SSC at about 45° C., followed by one or more washes in 0.2×SSC, 0.1% SDS at 60° C.; 3) high stringency hybridization conditions—6×SSC at about 45° C., followed by one or more washes in 0.2.×SSC, 0.1% SDS at 65° C.; and 4) very high stringency hybridization conditions—0.5M sodium phosphate, 7% SDS at 65° C., followed by one or more washes at 0.2×SSC, 1% SDS at 65° C. Very high stringency conditions (4) are the preferred conditions unless otherwise specified.

An “endogenous” polypeptide refers to a polypeptide encoded by the genome of the parental microbial cell (also termed “host cell”) from which the recombinant cell is engineered (or “derived”).

An “exogenous” polypeptide refers to a polypeptide which is not encoded by the genome of the parental microbial cell. A variant (i.e., mutant) polypeptide is an example of an exogenous polypeptide.

The term “heterologous” as used herein typically refers to a nucleotide sequence or a protein not naturally present in an organism. For example, a polynucleotide sequence endogenous to a plant can be introduced into a host cell by recombinant methods, and the plant polynucleotide is then a heterologous polynucleotide in a recombinant host cell.

As used herein, the term “fragment” of a polypeptide refers to a shorter portion of a full-length polypeptide or protein ranging in size from four amino acid residues to the entire amino acid sequence minus one amino acid residue. In certain embodiments of the invention, a fragment refers to the entire amino acid sequence of a domain of a polypeptide or protein (e.g., a substrate binding domain or a catalytic domain).

As used herein, the term “mutagenesis” refers to a process by which the genetic information of an organism is changed in a stable manner. Mutagenesis of a protein coding nucleic acid sequence produces a mutant protein. Mutagenesis also refers to changes in non-coding nucleic acid sequences that result in modified protein activity.

As used herein, the term “gene” refers to nucleic acid sequences encoding either an RNA product or a protein product, as well as operably-linked nucleic acid sequences affecting the expression of the RNA or protein (e.g., such sequences include but are not limited to promoter or enhancer sequences) or operably-linked nucleic acid sequences encoding sequences that affect the expression of the RNA or protein (e.g., such sequences include but are not limited to ribosome binding sites or translational control sequences).

Expression control sequences are known in the art and include, for example, promoters, enhancers, polyadenylation signals, transcription terminators, internal ribosome entry sites (IRES), and the like, that provide for the expression of the polynucleotide sequence in a host cell. Expression control sequences interact specifically with cellular proteins involved in transcription (Maniatis et al., Science, 236: 1237-1245 (1987)). Exemplary expression control sequences are described in, for example, Goeddel, Gene Expression Technology: Methods in Enzymology, Vol. 185, Academic Press, San Diego, Calif. (1990).

In the methods of the invention, an expression control sequence is operably linked to a polynucleotide sequence. By “operably linked” is meant that a polynucleotide sequence and an expression control sequence(s) are connected in such a way as to permit gene expression when the appropriate molecules (e.g., transcriptional activator proteins) are bound to the expression control sequence(s). Operably linked promoters are located upstream of the selected polynucleotide sequence in terms of the direction of transcription and translation. Operably linked enhancers can be located upstream, within, or downstream of the selected polynucleotide.

As used herein, the term “vector” refers to a nucleic acid molecule capable of transporting another nucleic acid, i.e., a polynucleotide sequence, to which it has been linked. One type of useful vector is an episome (i.e., a nucleic acid capable of extra-chromosomal replication). Useful vectors are those capable of autonomous replication and/or expression of nucleic acids to which they are linked. Vectors capable of directing the expression of genes to which they are operatively linked are referred to herein as “expression vectors.” In general, expression vectors of utility in recombinant DNA techniques are often in the form of “plasmids,” which refer generally to circular double stranded DNA loops that, in their vector form, are not bound to the chromosome. The terms “plasmid” and “vector” are used interchangeably herein, in as much as a plasmid is the most commonly used form of vector. However, also included are such other forms of expression vectors that serve equivalent functions and that become known in the art subsequently hereto.

In some embodiments, a recombinant vector further comprises a promoter operably linked to the polynucleotide sequence. In some embodiments, the promoter is a developmentally-regulated, an organelle-specific, a tissue-specific, an inducible, a constitutive, or a cell-specific promoter. The recombinant vector typically comprises at least one sequence selected from the group consisting of (a) an expression control sequence operatively coupled to the polynucleotide sequence; (b) a selection marker operatively coupled to the polynucleotide sequence; (c) a marker sequence operatively coupled to the polynucleotide sequence; (d) a purification moiety operatively coupled to the polynucleotide sequence; (e) a secretion sequence operatively coupled to the polynucleotide sequence; and (f) a targeting sequence operatively coupled to the polynucleotide sequence. In certain embodiments, the nucleotide sequence is stably incorporated into the genomic DNA of the host cell, and the expression of the nucleotide sequence is under the control of a regulated promoter region.

The expression vectors described herein include a polynucleotide sequence described herein in a form suitable for expression of the polynucleotide sequence in a host cell. It will be appreciated by those skilled in the art that the design of the expression vector can depend on such factors as the choice of the host cell to be transformed, the level of expression of polypeptide desired, etc. The expression vectors described herein can be introduced into host cells to produce polypeptides, including fusion polypeptides, encoded by the polynucleotide sequences as described herein.

Expression of genes encoding polypeptides in prokaryotes, for example, E. coli, is most often carried out with vectors containing constitutive or inducible promoters directing the expression of either fusion or non-fusion polypeptides. Fusion vectors add a number of amino acids to a polypeptide encoded therein, usually to the amino- or carboxy-terminus of the recombinant polypeptide. Such fusion vectors typically serve one or more of the following three purposes: (1) to increase expression of the recombinant polypeptide; (2) to increase the solubility of the recombinant polypeptide; and (3) to aid in the purification of the recombinant polypeptide by acting as a ligand in affinity purification. Often, in fusion expression vectors, a proteolytic cleavage site is introduced at the junction of the fusion moiety and the recombinant polypeptide. This enables separation of the recombinant polypeptide from the fusion moiety after purification of the fusion polypeptide. In certain embodiments, a polynucleotide sequence of the invention is operably linked to a promoter derived from bacteriophage T5.

In certain embodiments, the host cell is a yeast cell, and the expression vector is a yeast expression vector. Examples of vectors for expression in yeast S. cerevisiae include pYepSec1 (Baldari et al., EMBO J., 6: 229-234 (1987)), pMFa (Kurjan et al., Cell, 30: 933-943 (1982)), pJRY88 (Schultz et al., Gene, 54: 113-123 (1987)), pYES2 (Invitrogen Corp., San Diego, Calif.), and picZ (Invitrogen Corp., San Diego, Calif.).

In other embodiments, the host cell is an insect cell, and the expression vector is a baculovirus expression vector. Baculovirus vectors available for expression of proteins in cultured insect cells (e.g., Sf9 cells) include, for example, the pAc series (Smith et al., Mol. Cell. Biol., 3: 2156-2165 (1983)) and the pVL series (Lucklow et al., Virology, 170: 31-39 (1989)).

In yet another embodiment, the polynucleotide sequences described herein can be expressed in mammalian cells using a mammalian expression vector. Other suitable expression systems for both prokaryotic and eukaryotic cells are well known in the art; see, e.g., Sambrook et al., “Molecular Cloning: A Laboratory Manual,” second edition, Cold Spring Harbor Laboratory, (1989).

As used herein “acyl-CoA” refers to an acyl thioester formed between the carbonyl carbon of alkyl chain and the sulfhydryl group of the 4′-phosphopantethionyl moiety of coenzyme A (CoA), which has the formula R—C(O)S-CoA, where R is any alkyl group having at least 4 carbon atoms.

As used herein “acyl-ACP” refers to an acyl thioester formed between the carbonyl carbon of alkyl chain and the sulfhydryl group of the phosphopantetheinyl moiety of an acyl carrier protein (ACP). The phosphopantetheinyl moiety is post-translationally attached to a conserved serine residue on the ACP by the action of holo-acyl carrier protein synthase (ACPS), a phosphopantetheinyl transferase. In some embodiments an acyl-ACP is an intermediate in the synthesis of fully saturated acyl-ACPs. In other embodiments an acyl-ACP is an intermediate in the synthesis of unsaturated acyl-ACPs. In some embodiments, the carbon chain will have about 5, 6, 7, 8, 9, 10, 11, 12, 13, 14, 15, 16, 17, 18, 19, 20, 21, 22, 23, 24, 25, or 26 carbons. Each of these acyl-ACPs are substrates for enzymes that convert them to fatty acid derivatives.

As used herein, the term “fatty acid derivative” means a “fatty acid” or a “fatty acid derivative”, which may be referred to as a “fatty acid or derivative thereof”. The term “fatty acid” means a carboxylic acid having the formula RCOOH. R represents an aliphatic group, preferably an alkyl group. R can comprise between about 4 and about 22 carbon atoms. Fatty acids can be saturated, monounsaturated, or polyunsaturated. A “fatty acid derivative” is a product made in part from the fatty acid biosynthetic pathway of the production host organism. “Fatty acid derivatives” includes products made in part from acyl-ACP or acyl-ACP derivatives. Exemplary fatty acid derivatives include, for example, acyl-CoA, fatty acids, fatty aldehydes, short and long chain alcohols, hydrocarbons, fatty alcohols, esters (e.g., waxes, fatty acid esters, or fatty esters), terminal olefins, internal olefins, and ketones.

A “fatty acid derivative composition” as referred to herein is produced by a recombinant host cell and typically comprises a mixture of fatty acid derivative. In some cases, the mixture includes more than one type of product (e.g., fatty acids and fatty alcohols, fatty acids and fatty acid esters or alkanes and olefins). In other cases, the fatty acid derivative compositions may comprise, for example, a mixture of fatty esters (or another fatty acid derivative) with various chain lengths and saturation or branching characteristics. In still other cases, the fatty acid derivative composition comprises a mixture of both more than one type of product and products with various chain lengths and saturation or branching characteristics.

As used herein, the term “fatty acid biosynthetic pathway” means a biosynthetic pathway that produces fatty acids and derivatives thereof. The fatty acid biosynthetic pathway may include additional enzymes to produce fatty acids derivatives having desired characteristics.

As used herein, the term “fatty ester” means an ester. In a preferred embodiment, a fatty ester is any ester made from a fatty acid to produce, for example, a fatty acid ester. In one embodiment, a fatty ester contains an A side (i.e., the carbon chain attached to the carboxylate oxygen) and a B side (i.e., the carbon chain comprising the parent carboxylate). In a preferred embodiment, when the fatty ester is derived from the fatty acid biosynthetic pathway, the A side is contributed by an alcohol, and the B side is contributed by a fatty acid. Any alcohol can be used to form the A side of the fatty esters. For example, the alcohol can be derived from the fatty acid biosynthetic pathway. Alternatively, the alcohol can be produced through non-fatty acid biosynthetic pathways. Moreover, the alcohol can be provided exogenously. For example, the alcohol can be supplied in the fermentation broth in instances where the fatty ester is produced by an organism that can also produce the fatty acid. Alternatively, a carboxylic acid, such as a fatty acid or acetic acid, can be supplied exogenously in instances where the fatty ester is produced by an organism that can also produce alcohol.

The carbon chains comprising the A side or B side can be of any length. In one embodiment, the A side of the ester is at least about 1, 2, 3, 4, 5, 6, 7, 8, 10, 12, 14, 16, or 18 carbons in length. The B side of the ester is at least about 4, 6, 8, 10, 12, 14, 16, 18, 20, 22, 24, or 26 carbons in length. The A side and/or the B side can be straight or branched chain. The branched chains may have one or more points of branching. In addition, the branched chains may include cyclic branches. Furthermore, the A side and/or B side can be saturated or unsaturated. If unsaturated, the A side and/or B side can have one or more points of unsaturation.

In one embodiment, the fatty ester is produced biosynthetically. In this embodiment, first the fatty acid is “activated.” Non-limiting examples of “activated” fatty acids are acyl-CoA, acyl-ACP, and acyl phosphate. Acyl-CoA can be a direct product of fatty acid biosynthesis or degradation. In addition, acyl-CoA can be synthesized from a free fatty acid, a CoA, or an adenosine nucleotide triphosphate (ATP). An example of an enzyme which produces acyl-CoA is acyl-CoA synthase.

After the fatty acid is activated, it can be readily transferred to a recipient nucleophile. Exemplary nucleophiles are alcohols, thiols, or phosphates.

In one embodiment, the fatty ester is a wax. The wax can be derived from a long chain alcohol and a long chain fatty acid. In another embodiment, the fatty ester can be derived from a fatty acyl-thioester and an alcohol. In another embodiment, the fatty ester is a fatty acid thioester, for example fatty acyl Coenzyme A (CoA). In other embodiments, the fatty ester is a fatty acyl panthothenate, an acyl carrier protein (ACP), or a fatty phosphate ester. Fatty esters have many uses. For example, fatty esters can be used as biofuels, surfactants, or formulated into additives that provide lubrication and other benefits to fuels and industrial chemicals.

The R group of a fatty acid derivative, for example a fatty ester, can be a straight chain or a branched chain. Branched chains may have more than one point of branching and may include cyclic branches. In some embodiments, the branched fatty ester is a C6, C7, C8, C9, C10, C11, C12, C13, C14, C15, C16, C17, C18, C19, C20, C21, C22, C23, C24, C25, or a C26 branched fatty ester. In particular embodiments, the branched fatty acid, branched fatty aldehyde, or branched fatty alcohol is a C6, C8, C10, C12, C13, C14, C15, C16, C17, or C₁₋₈ branched fatty acid, branched fatty aldehyde, or branched fatty alcohol. In certain embodiments, the hydroxyl group of the branched fatty acid, branched fatty aldehyde, or branched fatty alcohol is in the primary (C1) position.

The R group of a branched or unbranched fatty ester derivative can be saturated or unsaturated. If unsaturated, the R group can have one or more than one point of unsaturation. In some embodiments, the unsaturated fatty acid derivative is a monounsaturated fatty acid derivative. In certain embodiments, the unsaturated fatty acid derivative is a C6:1, C7:1, C8:1, C9:1, C10:1, C11:1, C12:1, C13:1, C14:1, C15:1, C16:1, C17:1, C18:1, C19:1, C20:1, C21:1, C22:1, C23:1, C24:1, C25:1, or a C26:1 unsaturated fatty acid derivative. In certain embodiments, the unsaturated fatty ester, is a C10:1, C12:1, C14:1, C16:1, or C18:1 unsaturated fatty ester. In other embodiments, the unsaturated fatty ester is unsaturated at the omega-7 position. In certain embodiments, the unsaturated fatty ester comprises a cis double bond.

As used herein, a recombinant or engineered “host cell” is a host cell, e.g., a microorganism used to produce one or more of fatty esters including, for example, a fatty ester composition comprising one more types of esters (e.g., waxes, fatty acid esters, or fatty esters), together with beta-hydroxy esters.

In some embodiments, the recombinant host cell comprises one or more polynucleotides, each polynucleotide encoding a polypeptide having fatty acid biosynthetic enzyme activity, wherein the recombinant host cell produces a fatty ester composition when cultured in the presence of a carbon source under conditions effective to express the polynucleotides.

As used herein, the term “clone” typically refers to a cell or group of cells descended from and essentially genetically identical to a single common ancestor, for example, the bacteria of a cloned bacterial colony arose from a single bacterial cell.

As used herein, the term “culture” typical refers to a liquid media comprising viable cells. In one embodiment, a culture comprises cells reproducing in a predetermined culture media under controlled conditions, for example, a culture of recombinant host cells grown in liquid media comprising a selected carbon source and nitrogen.

“Culturing” or “cultivation” refers to growing a population of recombinant host cells under suitable conditions in a liquid or solid medium. In particular embodiments, culturing refers to the fermentative bioconversion of a substrate to an end-product. Culturing media are well known and individual components of such culture media are available from commercial sources, e.g., under the Difco™ and BBL™ trademarks. In one non-limiting example, the aqueous nutrient medium is a “rich medium” comprising complex sources of nitrogen, salts, and carbon, such as YP medium, comprising 10 g/L of peptone and 10 g/L yeast extract of such a medium.

The host cell can be additionally engineered to assimilate carbon efficiently and use cellulosic materials as carbon sources according to methods described in U.S. Pat. Nos. 5,000,000; 5,028,539; 5,424,202; 5,482,846; 5,602,030; WO 2010127318. In addition, in some embodiments the host cell is engineered to express an invertase so that sucrose can be used as a carbon source.

As used herein, the term “under conditions effective to express said heterologous nucleotide sequence(s)” means any conditions that allow a host cell to produce a desired fatty ester. Suitable conditions include, for example, fermentation conditions.

As used herein, “modified” or an “altered level of” activity of a protein, for example an enzyme, in a recombinant host cell refers to a difference in one or more characteristics in the activity determined relative to the parent or native host cell. Typically differences in activity are determined between a recombinant host cell, having modified activity, and the corresponding wild-type host cell (e.g., comparison of a culture of a recombinant host cell relative to the corresponding wild-type host cell). Modified activities can be the result of, for example, modified amounts of protein expressed by a recombinant host cell (e.g., as the result of increased or decreased number of copies of DNA sequences encoding the protein, increased or decreased number of mRNA transcripts encoding the protein, and/or increased or decreased amounts of protein translation of the protein from mRNA); changes in the structure of the protein (e.g., changes to the primary structure, such as, changes to the protein's coding sequence that result in changes in substrate specificity, changes in observed kinetic parameters); and changes in protein stability (e.g., increased or decreased degradation of the protein). In some embodiments, the polypeptide is a mutant or a variant of any of the polypeptides described herein. In certain instances, the coding sequence for the polypeptides described herein are codon optimized for expression in a particular host cell. For example, for expression in E. coli, one or more codons can be optimized as described in, e.g., Grosjean et al., Gene 18:199-209 (1982).

The term “regulatory sequences” as used herein typically refers to a sequence of bases in DNA, operably-linked to DNA sequences encoding a protein that ultimately controls the expression of the protein. Examples of regulatory sequences include, but are not limited to, RNA promoter sequences, transcription factor binding sequences, transcription termination sequences, modulators of transcription (such as enhancer elements), nucleotide sequences that affect RNA stability, and translational regulatory sequences (such as, ribosome binding sites (e.g., Shine-Dalgarno sequences in prokaryotes or Kozak sequences in eukaryotes), initiation codons, termination codons).

As used herein, the phrase “the expression of said nucleotide sequence is modified relative to the wild type nucleotide sequence,” means an increase or decrease in the level of expression and/or activity of an endogenous nucleotide sequence or the expression and/or activity of a heterologous or non-native polypeptide-encoding nucleotide sequence.

As used herein, the term “express” with respect to a polynucleotide is to cause it to function. A polynucleotide which encodes a polypeptide (or protein) will, when expressed, be transcribed and translated to produce that polypeptide (or protein). As used herein, the term “overexpress” means to express or cause to be expressed a polynucleotide or polypeptide in a cell at a greater concentration than is normally expressed in a corresponding wild-type cell under the same conditions.

The terms “altered level of expression” and “modified level of expression” are used interchangeably and mean that a polynucleotide, polypeptide, or hydrocarbon is present in a different concentration in an engineered host cell as compared to its concentration in a corresponding wild-type cell under the same conditions.

As used herein, the term “titer” refers to the quantity of fatty ester produced per unit volume of host cell culture. In any aspect of the compositions and methods described herein, a fatty ester is produced at a titer of about 25 mg/L, about 50 mg/L, about 75 mg/L, about 100 mg/L, about 125 mg/L, about 150 mg/L, about 175 mg/L, about 200 mg/L, about 225 mg/L, about 250 mg/L, about 275 mg/L, about 300 mg/L, about 325 mg/L, about 350 mg/L, about 375 mg/L, about 400 mg/L, about 425 mg/L, about 450 mg/L, about 475 mg/L, about 500 mg/L, about 525 mg/L, about 550 mg/L, about 575 mg/L, about 600 mg/L, about 625 mg/L, about 650 mg/L, about 675 mg/L, about 700 mg/L, about 725 mg/L, about 750 mg/L, about 775 mg/L, about 800 mg/L, about 825 mg/L, about 850 mg/L, about 875 mg/L, about 900 mg/L, about 925 mg/L, about 950 mg/L, about 975 mg/L, about 1000 mg/L, about 1050 mg/L, about 1075 mg/L, about 1100 mg/L, about 1125 mg/L, about 1150 mg/L, about 1175 mg/L, about 1200 mg/L, about 1225 mg/L, about 1250 mg/L, about 1275 mg/L, about 1300 mg/L, about 1325 mg/L, about 1350 mg/L, about 1375 mg/L, about 1400 mg/L, about 1425 mg/L, about 1450 mg/L, about 1475 mg/L, about 1500 mg/L, about 1525 mg/L, about 1550 mg/L, about 1575 mg/L, about 1600 mg/L, about 1625 mg/L, about 1650 mg/L, about 1675 mg/L, about 1700 mg/L, about 1725 mg/L, about 1750 mg/L, about 1775 mg/L, about 1800 mg/L, about 1825 mg/L, about 1850 mg/L, about 1875 mg/L, about 1900 mg/L, about 1925 mg/L, about 1950 mg/L, about 1975 mg/L, about 2000 mg/L (2 g/L), 3 g/L, 5 g/L, 10 g/L, 20 g/L, 30 g/L, 40 g/L, 50 g/L, 60 g/L, 70 g/L, 80 g/L, 90 g/L, 100 g/L or a range bounded by any two of the foregoing values. In other embodiments, a fatty ester is produced at a titer of more than 100 g/L, more than 200 g/L, more than 300 g/L, or higher, such as 500 g/L, 700 g/L or more. The preferred titer of fatty ester produced by a recombinant host cell according to the methods of the invention is from 5 g/L to 200 g/L, 10 g/L to 150 g/L, 20 g/L to 120 g/L and 30 g/L to 100 g/L. The titer may refer to a particular fatty ester or a combination of fatty esters produced by a given recombinant host cell culture.

As used herein, the “yield of fatty ester produced by a host cell” refers to the efficiency by which an input carbon source is converted to product (i.e., fatty esters) in a host cell. Host cells engineered to produce fatty esters according to the methods of the invention have a yield of at least 3%, at least 4%, at least 5%, at least 6%, at least 7%, at least 8%, at least 9%, at least 10%, at least 11%, at least 12%, at least 13%, at least 14%, at least 15%, at least 16%, at least 17%, at least 18%, at least 19%, at least 20%, at least 21%, at least 22%, at least 23%, at least 24%, at least 25%, at least 26%, at least 27%, at least 28%, at least 29%, or at least 30% or a range bounded by any two of the foregoing values. It is understood by those of skill in the art that the yield is dependent upon chain length. In other embodiments, a fatty ester or derivatives is produced at a yield of more than 30%, 40%, 50%, 60%, 70%, 80%, 90% or more. Alternatively, or in addition, the yield is about 30% or less, about 27% or less, about 25% or less, or about 22% or less. Thus, the yield can be bounded by any two of the above endpoints. For example, the yield of a fatty ester or fatty ester derivative produced by the recombinant host cell according to the methods of the invention can be 5% to 15%, 10% to 25%, 10% to 22%, 15% to 27%, 18% to 22%, 20% to 28%, or 20% to 30%. The yield may refer to a particular fatty ester or a combination of fatty esters produced by a given recombinant host cell culture.

As used herein, the term “productivity” refers to the quantity of a fatty ester or derivatives produced per unit volume of host cell culture per unit time. In any aspect of the compositions and methods described herein, the productivity of a fatty ester or derivatives produced by a recombinant host cell is at least 100 mg/L/hour, at least 200 mg/L/hour, at least 300 mg/L/hour, at least 400 mg/L/hour, at least 500 mg/L/hour, at least 600 mg/L/hour, at least 700 mg/L/hour, at least 800 mg/L/hour, at least 900 mg/L/hour, at least 1000 mg/L/hour, at least 1100 mg/L/hour, at least 1200 mg/L/hour, at least 1300 mg/L/hour, at least 1400 mg/L/hour, at least 1500 mg/L/hour, at least 1600 mg/L/hour, at least 1700 mg/L/hour, at least 1800 mg/L/hour, at least 1900 mg/L/hour, at least 2000 mg/L/hour, at least 2100 mg/L/hour, at least 2200 mg/L/hour, at least 2300 mg/L/hour, at least 2400 mg/L/hour, or at least 2500 mg/L/hour. Alternatively, or in addition, the productivity is 2500 mg/L/hour or less, 2000 mg/L/OD600 (“optical density at 600 nm”) or less, 1500 mg/L/OD600 or less, 120 mg/L/hour, or less, 1000 mg/L/hour or less, 800 mg/L/hour, or less, or 600 mg/L/hour or less. Thus, the productivity can be bounded by any two of the above endpoints. For example, the productivity can be 3 to 30 mg/L/hour, 6 to 20 mg/L/hour, or 15 to 30 mg/L/hour. For example, the productivity of a fatty ester or fatty ester derivative produced by a recombinant host cell according to the methods of the may be from 500 mg/L/hour to 2500 mg/L/hour, or from 700 mg/L/hour to 2000 mg/L/hour. The productivity may refer to a particular fatty ester or a combination of fatty esters produced by a given recombinant host cell culture.

As used herein, the term “total fatty species” generally means and fatty acids and fatty esters, as evaluated by GC-FID as described in International Patent Application Publication WO 2008/119082.

As used herein, the term “total fatty acid product” means FAME+FFA.

As used herein, the term “glucose utilization rate” means the amount of glucose used by a cell culture per unit time, typically reported as grams/liter/hour (g/L/hr).

As used herein, the term “carbon source” refers to a substrate or compound suitable to be used as a source of carbon for prokaryotic or simple eukaryotic cell growth. Carbon sources can be in various forms, including, but not limited to polymers, carbohydrates, acids, alcohols, aldehydes, ketones, amino acids, peptides, and gases (e.g., CO and CO2). Exemplary carbon sources include, but are not limited to, monosaccharides, such as glucose, fructose, mannose, galactose, xylose, and arabinose; oligosaccharides, such as fructo-oligosaccharide and galacto-oligosaccharide; polysaccharides such as starch, cellulose, pectin, and xylan; disaccharides, such as sucrose, maltose, cellobiose, and turanose; cellulosic material and variants such as hemicelluloses, methyl cellulose and sodium carboxymethyl cellulose; saturated or unsaturated fatty acids, succinate, lactate, and acetate; alcohols, such as ethanol, methanol, and glycerol, or mixtures thereof. The carbon source can also be a product of photosynthesis, such as glucose. In certain preferred embodiments, the carbon source is biomass. In other preferred embodiments, the carbon source is glucose. In other preferred embodiments the carbon source is sucrose.

As used herein, the term “biomass” refers to any biological material from which a carbon source is derived. In some embodiments, a biomass is processed into a carbon source, which is suitable for bioconversion. In other embodiments, the biomass does not require further processing into a carbon source. The carbon source can be converted into a biofuel. An exemplary source of biomass is plant matter or vegetation, such as corn, sugar cane, or switchgrass. Another exemplary source of biomass is metabolic waste products, such as animal matter (e.g., cow manure). Further exemplary sources of biomass include algae and other marine plants. Biomass also includes waste products from industry, agriculture, forestry, and households, including, but not limited to, fermentation waste, ensilage, straw, lumber, sewage, garbage, cellulosic urban waste, and food leftovers. The term “biomass” also can refer to sources of carbon, such as carbohydrates (e.g., monosaccharides, disaccharides, or polysaccharides).

As used herein, the term “isolated,” with respect to products (such as fatty acids and derivatives thereof) refers to products that are separated from cellular components, cell culture media, or chemical or synthetic precursors. The fatty acids and derivatives thereof produced by the methods described herein can be relatively immiscible in the fermentation broth, as well as in the cytoplasm. Therefore, the fatty acids and derivatives thereof can collect in an organic phase either intracellularly or extracellularly.

As used herein, the terms “purify,” “purified,” or “purification” mean the removal or isolation of a molecule from its environment by, for example, isolation or separation. “Substantially purified” molecules are at least about 60% free (e.g., at least about 70% free, at least about 75% free, at least about 85% free, at least about 90% free, at least about 95% free, at least about 97% free, at least about 99% free) from other components with which they are associated. As used herein, these terms also refer to the removal of contaminants from a sample. For example, the removal of contaminants can result in an increase in the percentage of fatty esters in a sample. For example, when a fatty ester is produced in a recombinant host cell, the fatty ester can be purified by the removal of host cell proteins. After purification, the percentage of fatty ester in the sample is increased. The terms “purify,” “purified,” and “purification” are relative terms which do not require absolute purity. Thus, for example, when a fatty ester is produced in recombinant host cells, a purified fatty ester is a fatty ester that is substantially separated from other cellular components (e.g., nucleic acids, polypeptides, lipids, carbohydrates, or other hydrocarbons).

### Generation of Fatty Acid Derivative by Recombinant Host Cells

This disclosure provides numerous examples of polypeptides (i.e., enzymes) having activities suitable for use in the fatty acid biosynthetic pathways described herein. Such polypeptides are collectively referred to herein as “fatty acid biosynthetic polypeptides” or “fatty acid biosynthetic enzymes”. Non-limiting examples of fatty acid pathway polypeptides suitable for use in recombinant host cells of the invention are provided herein.

In some embodiments, the invention includes a recombinant host cell comprising a polynucleotide sequence (also referred to herein as a “fatty acid biosynthetic polynucleotide” sequence) which encodes a fatty acid biosynthetic polypeptide.

The polynucleotide sequence, which comprises an open reading frame encoding a fatty acid biosynthetic polypeptide and operably-linked regulatory sequences, can be integrated into a chromosome of the recombinant host cells, incorporated in one or more plasmid expression systems resident in the recombinant host cell, or both. In the Examples, both plasmid expression systems and integration into the host genome are used to illustrate different embodiments of the present invention.

In some embodiments, a fatty acid biosynthetic polynucleotide sequence encodes a polypeptide which is endogenous to the parental host cell of the recombinant cell being engineered. Some such endogenous polypeptides are overexpressed in the recombinant host cell. In some embodiments, the fatty acid biosynthetic polynucleotide sequence encodes an exogenous or heterologous polypeptide. A variant (that is, a mutant) polypeptide is an example of a heterologous polypeptide.

In certain embodiments, the genetically modified host cell overexpresses a gene encoding a polypeptide (protein) that increases the rate at which the host cell produces the substrate of a fatty acid biosynthetic enzyme, i.e., a fatty acyl-thioester substrate. In certain embodiments, the enzyme encoded by the over expressed gene is directly involved in fatty acid biosynthesis.

Such recombinant host cells may be further engineered to comprise a polynucleotide sequence encoding one or more “fatty acid biosynthetic polypeptides”, (enzymes involved in fatty acid biosynthesis), for example, a polypeptide:

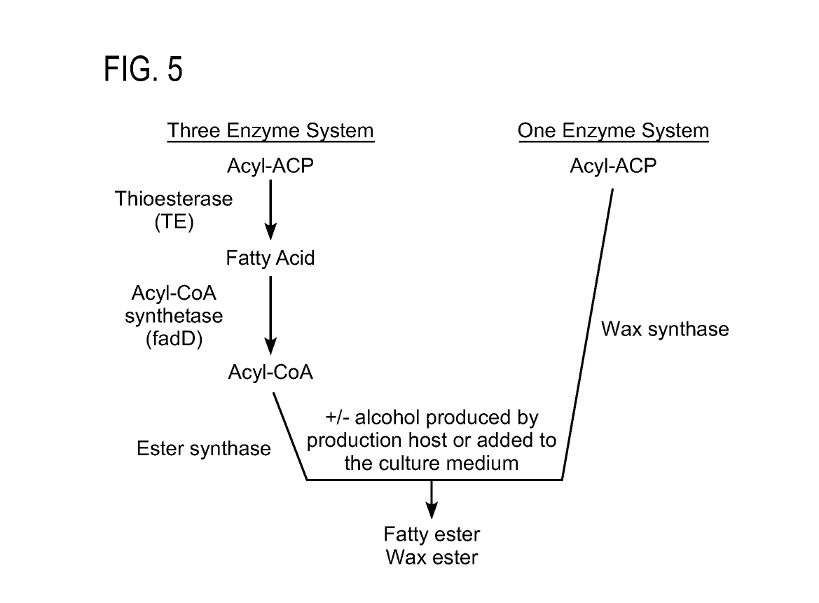

(1) having ester synthase activity wherein the recombinant host cell synthesizes fatty esters (“one enzyme system”; FIG. 5); or

(2) having thioesterase activity, acyl-CoA synthase activity and ester synthase activity wherein the recombinant host cell synthesizes fatty esters (“three enzyme system”; FIG. 5).

### Production of Fatty Esters

The recombinant host cells of the invention comprise one or more polynucleotide sequences that comprise an open reading frame encoding an ester synthase, e.g., any polypeptide which catalyzes the conversion of an acyl-thioester to a fatty ester, (for example, having an Enzyme Commission number of EC 2.3.1.75), together with operably-linked regulatory sequences that facilitate expression of the protein in the recombinant host cells. In the recombinant host cells, the open reading frame coding sequences and/or the regulatory sequences may be modified relative to the corresponding wild-type coding sequence of the ester synthase. A fatty ester composition comprising beta hydroxy esters is produced by culturing a recombinant cell in the presence of a carbon source under conditions effective to express the ester synthase. Expression of different ester synthases and mutants or variants thereof will result in production of differing amounts of beta-hydroxy esters in combination with the corresponding ester which lacks the beta-hydroxy moiety.

In related embodiments, the recombinant host cell comprises a polynucleotide encoding a polypeptide having ester synthase activity, and one or more additional polynucleotides encoding polypeptides having other fatty ester biosynthetic enzyme activities.

As used herein, the term “fatty ester” may be used with reference to an ester. A fatty ester as referred to herein can be any ester made from a fatty acid, for example a fatty acid ester. In some embodiments, a fatty ester contains an A side and a B side. As used herein, an “A side” of an ester refers to the carbon chain attached to the carboxylate oxygen of the ester. As used herein, a “B side” of an ester refers to the carbon chain comprising the parent carboxylate of the ester. In embodiments where the fatty ester is derived from the fatty acid biosynthetic pathway, the A side is contributed by an alcohol, and the B side is contributed by a fatty acid.

Any alcohol can be used to form the A side of the fatty esters. For example, the alcohol can be derived from the fatty acid biosynthetic pathway, such as those describe hereinabove. Alternatively, the alcohol can be produced through non-fatty acid biosynthetic pathways. Moreover, the alcohol can be provided exogenously. For example, the alcohol can be supplied in the fermentation broth in instances where the fatty ester is produced by an organism. Alternatively, a carboxylic acid, such as a fatty acid or acetic acid, can be supplied exogenously in instances where the fatty ester is produced by an organism that can also produce alcohol.

The carbon chains comprising the A side or B side can be of any length. In one embodiment, the A side of the ester is at least about 1, 2, 3, 4, 5, 6, 7, 8, 10, 12, 14, 16, or 18 carbons in length. When the fatty ester is a fatty acid methyl ester, the A side of the ester is 1 carbon in length. When the fatty ester is a fatty acid ethyl ester, the A side of the ester is 2 carbons in length. The B side of the ester can be at least about 4, 6, 8, 10, 12, 14, 16, 18, 20, 22, 24, or 26 carbons in length. The A side and/or the B side can be straight or branched chain. The branched chains can have one or more points of branching. In addition, the branched chains can include cyclic branches. Furthermore, the A side and/or B side can be saturated or unsaturated. If unsaturated, the A side and/or B side can have one or more points of unsaturation.

In one embodiment, the fatty ester is produced biosynthetically. In this embodiment, first the fatty acid is “activated.” Non-limiting examples of “activated” fatty acids are acyl-CoA, acyl ACP, and acyl phosphate. Acyl-CoA can be a direct product of fatty acid biosynthesis or degradation. In addition, acyl-CoA can be synthesized from a free fatty acid, a CoA, and an adenosine nucleotide triphosphate (ATP). An example of an enzyme which produces acyl-CoA is acyl-CoA synthase.

In some embodiments, the recombinant host cell comprises a polynucleotide encoding a polypeptide, e.g., an enzyme having ester synthase activity, (also referred to herein as an “ester synthase polypeptide” or an “ester synthase”). A fatty ester is produced by a reaction catalyzed by the ester synthase polypeptide expressed or overexpressed in the recombinant host cell. In some embodiments, a composition comprising fatty esters (also referred to herein as a “fatty ester composition”) comprising fatty esters is produced by culturing the recombinant cell in the presence of a carbon source under conditions effective to express an ester synthase. In some embodiments, the fatty ester composition is recovered from the cell culture.

Ester synthase polypeptides include, for example, an ester synthase polypeptide classified as EC 2.3.1.75, or any other polypeptide which catalyzes the conversion of an acyl-thioester to a fatty ester, including, without limitation, an ester synthase, an acyl-CoA:alcohol transacylase, an acyltransferase, or a fatty acyl-CoA:fatty alcohol acyltransferase. For example, the polynucleotide may encode wax/dgat, a bifunctional ester synthase/acyl-CoA:diacylglycerol acyltransferase from Simmondsia chinensis, Acinetobacter sp. Strain ADP, Alcanivoraxborkumensis, Pseudomonas aeruginosa, Fundibacter jadensis, Arabidopsis thaliana, or Alkaligeneseutrophus. In a particular embodiment, the ester synthase polypeptide is an Acinetobacter sp. diacylglycerol O-acyltransferase (wax-dgaT; UniProtKB Q8GGG1, GenBank AAO17391) or Simmondsia chinensis wax synthase (UniProtKB Q9XGY6, GenBank AAD38041. In another embodiment, the ester synthase polypeptide is for example ES9 (an ester synthase from Marinobacter hydrocarbonoclasticus DSM 8798, UniProtKB A3RE51; GenBank ABO21021, encoded by the WS2 gene; or ES376 (another ester ester synthase derived from Marinobacter hydrocarbonoclasticus DSM 8798, UniProtKB A3RE50, GenBank ABO21020, encoded by the wsl gene. In a particular embodiment, the polynucleotide encoding the ester synthase polypeptide is overexpressed in the recombinant host cell.

In some embodiments, a fatty acid ester is produced by a recombinant host cell engineered to express three fatty acid biosynthetic enzymes: a thioesterase enzyme, an acyl-CoA synthetase (fadD) enzyme and an ester synthase enzyme (“three enzyme system”; FIG. 5).

In other embodiments, a fatty acid ester is produced by a recombinant host cell engineered to express one fatty acid biosynthetic enzyme, an ester synthase enzyme (“one enzyme system”; FIG. 5).

Non-limiting examples of ester synthase polypeptides and polynucleotides encoding them suitable for use in these embodiments include those described in PCT Publication Nos. WO 2007/136762 and WO2008/119082, and WO/2011/038134 (“three enzyme system”) and WO/2011/038132 (“one enzyme system”).

The recombinant host cell may produce a fatty ester, such as a fatty acid methyl ester, a fatty acid ethyl ester or a wax ester in the extracellular environment of the host cells.

In some embodiments, the chain length of a fatty ester can be selected for by modifying the expression of particular thioesterases. The thioesterase will influence the chain length of fatty acid derivatives produced. The chain length of a fatty acid derivative substrate can be selected for by modifying the expression of selected thioesterases (EC 3.1.2.14 or EC 3.1.1.5). Hence, host cells can be engineered to express, overexpress, have attenuated expression, or not express one or more selected thioesterases to increase the production of a preferred fatty acid derivative substrate. For example, C₁₀ fatty acids can be produced by expressing a thioesterase that has a preference for producing C₁₀ fatty acids and attenuating thioesterases that have a preference for producing fatty acids other than C₁₀ fatty acids (e.g., a thioesterase which prefers to produce C₁₄ fatty acids). This would result in a relatively homogeneous population of fatty acids that have a carbon chain length of 10. In other instances, C₁₄ fatty acids can be produced by attenuating endogenous thioesterases that produce non-C₁₄ fatty acids and expressing the thioesterases that use C₁₄-ACP. In some situations, C₁₂ fatty acids can be produced by expressing thioesterases that use C₁₂-ACP and attenuating thioesterases that produce non-C₁₂ fatty acids. For example, C12 fatty acids can be produced by expressing a thioesterase that has a preference for producing C12 fatty acids and attenuating thioesterases that have a preference for producing fatty acids other than C12 fatty acids. This would result in a relatively homogeneous population of fatty acids that have a carbon chain length of 12. The fatty acid derivatives are recovered from the culture medium with substantially all of the fatty acid derivatives produced extracellularly. The fatty acid derivative composition produced by a recombinant host cell can be analyzed using methods known in the art, for example, GC-FID, in order to determine the distribution of particular fatty acid derivatives as well as chain lengths and degree of saturation of the components of the fatty acid derivative composition. Acetyl-CoA, malonyl-CoA, and fatty acid overproduction can be verified using methods known in the art, for example, by using radioactive precursors, HPLC, or GC-MS subsequent to cell lysis.

Non-limiting examples of thioesterases and polynucleotides encoding them for use in the fatty acid pathway are provided in PCT Publication No. WO 2010/075483.

### Production of Fatty Ester Compositions by Recombinant Host Cells

In some embodiments of the present invention, a high titer of fatty esters in a particular composition is a higher titer of a particular type of fatty acid derivative (e.g., fatty esters or beta-hydroxy fatty esters, or both) produced by a recombinant host cell culture relative to the titer of the same fatty acid derivatives produced by a control culture of a corresponding wild-type host cell.

In some embodiments, a polynucleotide (or gene) sequence is provided to the host cell by way of a recombinant vector, which comprises a promoter operably linked to the polynucleotide sequence. In certain embodiments, the promoter is a developmentally-regulated, an organelle-specific, a tissue-specific, an inducible, a constitutive, or a cell-specific promoter.

In some embodiments, the recombinant vector comprises at least one sequence selected from the group consisting of (a) an expression control sequence operatively coupled to the polynucleotide sequence; (b) a selection marker operatively coupled to the polynucleotide sequence; (c) a marker sequence operatively coupled to the polynucleotide sequence; (d) a purification moiety operatively coupled to the polynucleotide sequence; (e) a secretion sequence operatively coupled to the polynucleotide sequence; and (f) a targeting sequence operatively coupled to the polynucleotide sequence.

The expression vectors described herein include a polynucleotide sequence described herein in a form suitable for expression of the polynucleotide sequence in a host cell. It will be appreciated by those skilled in the art that the design of the expression vector can depend on such factors as the choice of the host cell to be transformed, the level of expression of polypeptide desired, etc. The expression vectors described herein can be introduced into host cells to produce polypeptides, including fusion polypeptides, encoded by the polynucleotide sequences as described herein.

Expression of genes encoding polypeptides in prokaryotes, for example, E. coli, is most often carried out with vectors containing constitutive or inducible promoters directing the expression of either fusion or non-fusion polypeptides. Fusion vectors add a number of amino acids to a polypeptide encoded therein, usually to the amino- or carboxy-terminus of the recombinant polypeptide. Such fusion vectors typically serve one or more of the following three purposes: (1) to increase expression of the recombinant polypeptide; (2) to increase the solubility of the recombinant polypeptide; and (3) to aid in the purification of the recombinant polypeptide by acting as a ligand in affinity purification. Often, in fusion expression vectors, a proteolytic cleavage site is introduced at the junction of the fusion moiety and the recombinant polypeptide. This enables separation of the recombinant polypeptide from the fusion moiety after purification of the fusion polypeptide. Examples of such enzymes, and their cognate recognition sequences, include Factor Xa, thrombin, and enterokinase. Exemplary fusion expression vectors include pGEX (Pharmacia Biotech, Inc., Piscataway, N.J.; Smith et al., Gene, 67: 31-40 (1988)), pMAL (New England Biolabs, Beverly, Mass.), and pRITS (Pharmacia Biotech, Inc., Piscataway, N.J.), which fuse glutathione S-transferase (GST), maltose E binding protein, or protein A, respectively, to the target recombinant polypeptide.

Examples of inducible, non-fusion E. coli expression vectors include pTrc (Amann et al., Gene (1988) 69:301-315) and pET 11d (Studier et al., Gene Expression Technology: Methods in Enzymology 185, Academic Press, San Diego, Calif. (1990) 60-89). Target gene expression from the pTrc vector relies on host RNA polymerase transcription from a hybrid trp-lac fusion promoter. Target gene expression from the pET 11d vector relies on transcription from a T7 gn10-lac fusion promoter mediated by a coexpressed viral RNA polymerase (T7 gn1). This viral polymerase is supplied by host strains BL21(DE3) or HMS174(DE3) from a resident λ prophage harboring a T7 gn1 gene under the transcriptional control of the lacUV 5 promoter.

Suitable expression systems for both prokaryotic and eukaryotic cells are well known in the art; see, e.g., Sambrook et al., “Molecular Cloning: A Laboratory Manual,” second edition, Cold Spring Harbor Laboratory, (1989). Examples of inducible, non-fusion E. coli expression vectors include pTrc (Amann et al., Gene, 69: 301-315 (1988)) and PET 11d (Studier et al., Gene Expression Technology Methods in Enzymology 185, Academic Press, San Diego, Calif., pp. 60-89 (1990)). In certain embodiments, a polynucleotide sequence of the invention is operably linked to a promoter derived from bacteriophage T5.

In one embodiment, the host cell is a yeast cell. In this embodiment, the expression vector is a yeast expression vector.

Vectors can be introduced into prokaryotic or eukaryotic cells via a variety of art-recognized techniques for introducing foreign nucleic acid (e.g., DNA) into a host cell. Suitable methods for transforming or transfecting host cells can be found in, for example, Sambrook et al. (supra).

For stable transformation of bacterial cells, it is known that, depending upon the expression vector and transformation technique used, only a small fraction of cells will take-up and replicate the expression vector. In order to identify and select these transformants, a gene that encodes a selectable marker (e.g., resistance to an antibiotic) can be introduced into the host cells along with the gene of interest. Selectable markers include those that confer resistance to drugs such as, but not limited to, ampicillin, kanamycin, chloramphenicol, or tetracycline. Nucleic acids encoding a selectable marker can be introduced into a host cell on the same vector as that encoding a polypeptide described herein or can be introduced on a separate vector. Cells stably transformed with the introduced nucleic acid can be identified by growth in the presence of an appropriate selection drug.

As used herein, the term “recombinant host cell” or “engineered host cell” refers to a host cell whose genetic makeup has been altered relative to the corresponding wild-type host cell, for example, by deliberate introduction of new genetic elements and/or deliberate modification of genetic elements naturally present in the host cell. The offspring of such recombinant host cells also contain these new and/or modified genetic elements. In any of the aspects of the invention described herein, the host cell can be selected from the group consisting of a plant cell, insect cell, fungus cell (e.g., a filamentous fungus, such as Candida sp., or a budding yeast, such as Saccharomyces sp.), an algal cell and a bacterial cell. In one preferred embodiment, recombinant host cells are “recombinant microorganisms” or “recombinant microbial cells”.

Examples of host cells that are microorganisms, include but are not limited to cells from the genus Escherichia, Bacillus, Lactobacillus, Zymomonas, Rhodococcus, Pseudomonas, Aspergillus, Trichoderma, Neurospora, Fusarium, Humicola, Rhizomucor, Kluyveromyces, Pichia, Mucor, Myceliophtora, Penicillium, Phanerochaete, Pleurotus, Trametes, Chrysosporium, Saccharomyces, Stenotrophamonas, Schizosaccharomyces, Yarrowia, or Streptomyces. In some embodiments, the host cell is a Gram-positive bacterial cell. In other embodiments, the host cell is a Gram-negative bacterial cell.

In some embodiments, the host cell is an E. coli cell.

In other embodiments, the host cell is a Bacillus lentus cell, a Bacillus brevis cell, a Bacillus stearothermophilus cell, a Bacillus lichenoformis cell, a Bacillus alkalophilus cell, a Bacillus coagulans cell, a Bacillus circulans cell, a Bacillus pumilis cell, a Bacillus thuringiensis cell, a Bacillus clausii cell, a Bacillus megaterium cell, a Bacillus subtilis cell, or a Bacillus amyloliquefaciens cell.

In other embodiments, the host cell is a Trichoderma koningii cell, a Trichoderma viride cell, a Trichoderma reesei cell, a Trichoderma longibrachiatum cell, an Aspergillus awamori cell, an Aspergillus fumigates cell, an Aspergillus foetidus cell, an Aspergillus nidulans cell, an Aspergillus niger cell, an Aspergillus oryzae cell, a Humicola insolens cell, a Humicola lanuginose cell, a Rhodococcus opacus cell, a Rhizomucor miehei cell, or a Mucor michei cell.

In yet other embodiments, the host cell is a Streptomyces lividans cell or a Streptomyces murinus cell.

In yet other embodiments, the host cell is an Actinomycetes cell.

In some embodiments, the host cell is a Saccharomyces cerevisiae cell. In some embodiments, the host cell is a Saccharomyces cerevisiae cell.

In other embodiments, the host cell is a cell from a eukaryotic plant, algae, cyanobacterium, green-sulfur bacterium, green non-sulfur bacterium, purple sulfur bacterium, purple non-sulfur bacterium, extremophile, yeast, fungus, an engineered organism thereof, or a synthetic organism. In some embodiments, the host cell is light-dependent or fixes carbon. In some embodiments, the host cell is light-dependent or fixes carbon. In some embodiments, the host cell has autotrophic activity. In some embodiments, the host cell has photoautotrophic activity, such as in the presence of light. In some embodiments, the host cell is heterotrophic or mixotrophic in the absence of light. In certain embodiments, the host cell is a cell from Arabidopsis thaliana, Panicum virgatum, Miscanthus giganteus, Zea mays, Botryococcuse braunii, Chlamydomonas reinhardtii, Dunaliela salina, Synechococcus Sp. PCC 7002, Synechococcus Sp. PCC 7942, Synechocystis Sp. PCC 6803, Thermosynechococcus elongates BP-1, Chlorobium tepidum, Chlorojlexus auranticus, Chromatiumm vinosum, Rhodospirillum rubrum, Rhodobacter capsulatus, Rhodopseudomonas palusris, Clostridium ljungdahlii, Clostridiuthermocellum, Penicillium chrysogenum, Pichia pastoris, Saccharomyces cerevisiae, Schizosaccharomyces pombe, Pseudomonas fluorescens, or Zymomonas mobilis.

### Mutants or Variants

In some embodiments, the polypeptide is a mutant or a variant of any of the polypeptides described herein. The terms “mutant” and “variant” as used herein refer to a polypeptide having an amino acid sequence that differs from a wild-type polypeptide by at least one amino acid. For example, the mutant can comprise one or more of the following conservative amino acid substitutions: replacement of an aliphatic amino acid, such as alanine, valine, leucine, and isoleucine, with another aliphatic amino acid; replacement of a serine with a threonine; replacement of a threonine with a serine; replacement of an acidic residue, such as aspartic acid and glutamic acid, with another acidic residue; replacement of a residue bearing an amide group, such as asparagine and glutamine, with another residue bearing an amide group; exchange of a basic residue, such as lysine and arginine, with another basic residue; and replacement of an aromatic residue, such as phenylalanine and tyrosine, with another aromatic residue. In some embodiments, the mutant polypeptide has about 1, 2, 3, 4, 5, 6, 7, 8, 9, 10, 15, 20, 30, 40, 50, 60, 70, 80, 90, 100, or more amino acid substitutions, additions, insertions, or deletions.

Preferred fragments or mutants of a polypeptide retain some or all of the biological function (e.g., enzymatic activity) of the corresponding wild-type polypeptide. In some embodiments, the fragment or mutant retains at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% or more of the biological function of the corresponding wild-type polypeptide. In other embodiments, the fragment or mutant retains about 100% of the biological function of the corresponding wild-type polypeptide. Guidance in determining which amino acid residues may be substituted, inserted, or deleted without affecting biological activity may be found using computer programs well known in the art, for example, LASERGENE™ software (DNASTAR, Inc., Madison, Wis.).

In yet other embodiments, a fragment or mutant exhibits increased biological function as compared to a corresponding wild-type polypeptide. For example, a fragment or mutant may display at least a 10%, at least a 25%, at least a 50%, at least a 60%, at least a 70%, at least a 75%, at least a 80%, at least a 85%, at least a 90%, or at least a 95% improvement in enzymatic activity as compared to the corresponding wild-type polypeptide. In other embodiments, the fragment or mutant displays at least 100% (e.g., at least 200%, or at least 500%) improvement in enzymatic activity as compared to the corresponding wild-type polypeptide.

It is understood that the polypeptides described herein may have additional conservative or non-essential amino acid substitutions, which do not have a substantial effect on the polypeptide function. Whether or not a particular substitution will be tolerated (i.e., will not adversely affect desired biological function, such as ester synthase activity) can be determined as described in Bowie et al. (Science, 247: 1306-1310 (1990)). A “conservative amino acid substitution” is one in which the amino acid residue is replaced with an amino acid residue having a similar side chain. Families of amino acid residues having similar side chains have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine), and aromatic side chains (e.g., tyrosine, phenylalanine, tryptophan, histidine).

Variants can be naturally occurring or created in vitro. In particular, such variants can be created using genetic engineering techniques, such as site directed mutagenesis, random chemical mutagenesis, Exonuclease III deletion procedures, or standard cloning techniques. Alternatively, such variants, fragments, analogs, or derivatives can be created using chemical synthesis or modification procedures.

Methods of making variants are well known in the art. These include procedures in which nucleic acid sequences obtained from natural isolates are modified to generate nucleic acids that encode polypeptides having characteristics that enhance their value in industrial or laboratory applications. In such procedures, a large number of variant sequences having one or more nucleotide differences with respect to the sequence obtained from the natural isolate are generated and characterized. Typically, these nucleotide differences result in amino acid changes with respect to the polypeptides encoded by the nucleic acids from the natural isolates.

For example, variants can be prepared by using random and site-directed mutagenesis. Random and site-directed mutagenesis are described in, for example, Arnold, Curr. Opin. Biotech., 4: 450-455 (1993).

Random mutagenesis can be achieved using error prone PCR (see, e.g., Leung et al., Technique, 1: 11-15 (1989); and Caldwell et al., PCR Methods Applic., 2: 28-33 (1992)). In error prone PCR, PCR is performed under conditions where the copying fidelity of the DNA polymerase is low, such that a high rate of point mutations is obtained along the entire length of the PCR product. Briefly, in such procedures, nucleic acids to be mutagenized (e.g., a polynucleotide sequence encoding an ester synthase enzyme) are mixed with PCR primers, reaction buffer, MgCl₂, MnCl₂, Taq polymerase, and an appropriate concentration of dNTPs for achieving a high rate of point mutation along the entire length of the PCR product. For example, the reaction can be performed using 20 fmoles of nucleic acid to be mutagenized, 30 pmole of each PCR primer, a reaction buffer comprising 50 mM KCl, 10 mM Tris HCl (pH 8.3), 0.01% gelatin, 7 mM MgCl₂, 0.5 mM MnCl₂, 5 units of Taq polymerase, 0.2 mM dGTP, 0.2 mM dATP, 1 mM dCTP, and 1 mM dTTP. PCR can be performed for 30 cycles of 94° C. for 1 min, 45° C. for 1 min, and 72° C. for 1 min. However, it will be appreciated that these parameters can be varied as appropriate. The mutagenized nucleic acids are then cloned into an appropriate vector, and the activities of the polypeptides encoded by the mutagenized nucleic acids are evaluated.

Site-directed mutagenesis can be achieved using oligonucleotide-directed mutagenesis to generate site-specific mutations in any cloned DNA of interest. Oligonucleotide mutagenesis is described in, for example, Reidhaar-Olson et al., Science, 241: 53-57 (1988). Briefly, in such procedures a plurality of double stranded oligonucleotides bearing one or more mutations to be introduced into the cloned DNA are synthesized and inserted into the cloned DNA to be mutagenized (e.g., a polynucleotide sequence encoding an ester synthase polypeptide). Clones containing the mutagenized DNA are recovered, and the activities of the polypeptides they encode are assessed.

Another method for generating variants is assembly PCR. Assembly PCR involves the assembly of a PCR product from a mixture of small DNA fragments. A large number of different PCR reactions occur in parallel in the same vial, with the products of one reaction priming the products of another reaction. Assembly PCR is described in, for example, U.S. Pat. No. 5,965,408.

Still another method of generating variants is sexual PCR mutagenesis. In sexual PCR mutagenesis, forced homologous recombination occurs between DNA molecules of different, but highly related, DNA sequences in vitro as a result of random fragmentation of the DNA molecule based on sequence homology. This is followed by fixation of the crossover by primer extension in a PCR reaction. Sexual PCR mutagenesis is described in, for example, Stemmer, Proc. Natl. Acad. Sci., U.S.A., 91: 10747-10751 (1994).

Variants can also be created by in vivo mutagenesis. In some embodiments, random mutations in a nucleic acid sequence are generated by propagating the sequence in a bacterial strain, such as an E. coli strain, which carries mutations in one or more of the DNA repair pathways. Such “mutator” strains have a higher random mutation rate than that of a wild-type strain. Propagating a DNA sequence (e.g., a polynucleotide sequence encoding an ester synthase polypeptide) in one of these strains will eventually generate random mutations within the DNA. Mutator strains suitable for use for in vivo mutagenesis are described in, for example, International Patent Application Publication No. WO 1991/016427.

Variants can also be generated using cassette mutagenesis. In cassette mutagenesis, a small region of a double-stranded DNA molecule is replaced with a synthetic oligonucleotide “cassette” that differs from the native sequence. The oligonucleotide often contains a completely and/or partially randomized native sequence.

Recursive ensemble mutagenesis can also be used to generate variants. Recursive ensemble mutagenesis is an algorithm for protein engineering (i.e., protein mutagenesis) developed to produce diverse populations of phenotypically related mutants whose members differ in amino acid sequence. This method uses a feedback mechanism to control successive rounds of combinatorial cassette mutagenesis. Recursive ensemble mutagenesis is described in, for example, Arkin et al., Proc. Natl. Acad. Sci., U.S.A., 89: 7811-7815 (1992).

In some embodiments, variants are created using exponential ensemble mutagenesis. Exponential ensemble mutagenesis is a process for generating combinatorial libraries with a high percentage of unique and functional mutants, wherein small groups of residues are randomized in parallel to identify, at each altered position, amino acids which lead to functional proteins. Exponential ensemble mutagenesis is described in, for example, Delegrave et al., Biotech. Res, 11: 1548-1552 (1993).

In some embodiments, variants are created using shuffling procedures wherein portions of a plurality of nucleic acids that encode distinct polypeptides are fused together to create chimeric nucleic acid sequences that encode chimeric polypeptides as described in, for example, U.S. Pat. Nos. 5,965,408 and 5,939,250.

Insertional mutagenesis is mutagenesis of DNA by the insertion of one or more bases. Insertional mutations can occur naturally, mediated by virus or transposon, or can be artificially created for research purposes in the lab, e.g., by transposon mutagenesis. When exogenous DNA is integrated into that of the host, the severity of any ensuing mutation depends entirely on the location within the host's genome wherein the DNA is inserted. For example, significant effects may be evident if a transposon inserts in the middle of an essential gene, in a promoter region, or into a repressor or an enhancer region. Transposon mutagenesis and high-throughput screening was done to find beneficial mutations that increase the titer or yield of a fatty acid derivative or derivatives.

### Culture Recombinant Host Cells and Cell Cultures/Fermentation

As used herein, the term “fermentation” broadly refers to the conversion of organic materials into target substances by host cells, for example, the conversion of a carbon source by recombinant host cells into fatty acids or derivatives thereof by propagating a culture of the recombinant host cells in a media comprising the carbon source.

As used herein, the term “conditions permissive for the production” means any conditions that allow a host cell to produce a desired product, such as a fatty acid ester composition comprising a beta-hydroxy ester. Similarly, the term “conditions in which the polynucleotide sequence of a vector is expressed” means any conditions that allow a host cell to synthesize a polypeptide. Suitable conditions include, for example, fermentation conditions. Fermentation conditions can comprise many parameters, including but not limited to temperature ranges, levels of aeration, feed rates and media composition. Each of these conditions, individually and in combination, allows the host cell to grow. Fermentation can be aerobic, anaerobic, or variations thereof (such as micro-aerobic). Exemplary culture media include broths or gels. Generally, the medium includes a carbon source that can be metabolized by a host cell directly. In addition, enzymes can be used in the medium to facilitate the mobilization (e.g., the depolymerization of starch or cellulose to fermentable sugars) and subsequent metabolism of the carbon source.

For small scale production, the engineered host cells can be grown in batches of, for example, about 100 mL, 500 mL, 1 L, 2 L, 5 L, or 10 L; fermented; and induced to express a desired polynucleotide sequence, such as a polynucleotide sequence encoding an ester synthase polypeptide. For large scale production, the engineered host cells can be grown in batches of about 10 L, 100 L, 1000 L, 10,000 L, 100,000 L, 1,000,000 L or larger; fermented; and induced to express a desired polynucleotide sequence.

The fatty ester compositions described herein are found in the extracellular environment of the recombinant host cell culture and can be readily isolated from the culture medium. A fatty acid derivative may be secreted by the recombinant host cell, transported into the extracellular environment or passively transferred into the extracellular environment of the recombinant host cell culture. The fatty ester composition may be isolated from a recombinant host cell culture using routine methods known in the art.

Products Derived from Recombinant Host Cells

As used herein, “fraction of modem carbon” or fM has the same meaning as defined by National Institute of Standards and Technology (NIST) Standard Reference Materials (SRMs4990B and 4990C, known as oxalic acids standards HOxI and HOxII, respectively. The fundamental definition relates to 0.95 times the 14C/12C isotope ratio HOxI (referenced to AD 1950). This is roughly equivalent to decay-corrected pre-Industrial Revolution wood. For the current living biosphere (plant material), fM is approximately 1.1.

Bioproducts (e.g., the fatty ester compositions produced in accordance with the present disclosure) comprising biologically produced organic compounds, and in particular, the fatty ester compositions produced using the fatty acid biosynthetic pathway herein, have not been produced from renewable sources and, as such, are new compositions of matter. These new bioproducts can be distinguished from organic compounds derived from petrochemical carbon on the basis of dual carbon-isotopic fingerprinting or ¹⁴C dating. Additionally, the specific source of biosourced carbon (e.g., glucose vs. glycerol) can be determined by dual carbon-isotopic fingerprinting (see, e.g., U.S. Pat. No. 7,169,588, which is herein incorporated by reference).

The ability to distinguish bioproducts from petroleum based organic compounds is beneficial in tracking these materials in commerce. For example, organic compounds or chemicals comprising both biologically based and petroleum based carbon isotope profiles may be distinguished from organic compounds and chemicals made only of petroleum based materials. Hence, the bioproducts herein can be followed or tracked in commerce on the basis of their unique carbon isotope profile.

Bioproducts can be distinguished from petroleum based organic compounds by comparing the stable carbon isotope ratio (¹³C/¹²C) in each sample. The ¹³C/¹²C ratio in a given bioproduct is a consequence of the ¹³C/¹²C ratio in atmospheric carbon dioxide at the time the carbon dioxide is fixed. It also reflects the precise metabolic pathway. Regional variations also occur. Petroleum, C3 plants (the broadleaf), C4 plants (the grasses), and marine carbonates all show significant differences in ¹³C/¹²C and the corresponding δ¹³C values. Furthermore, lipid matter of C3 and C4 plants analyze differently than materials derived from the carbohydrate components of the same plants as a consequence of the metabolic pathway. Within the precision of measurement, ¹³C shows large variations due to isotopic fractionation effects, the most significant of which for bioproducts is the photosynthetic mechanism. The major cause of differences in the carbon isotope ratio in plants is closely associated with differences in the pathway of photosynthetic carbon metabolism in the plants, particularly the reaction occurring during the primary carboxylation (i.e., the initial fixation of atmospheric CO₂). Two large classes of vegetation are those that incorporate the “C3” (or Calvin-Benson) photosynthetic cycle and those that incorporate the “C4” (or Hatch-Slack) photosynthetic cycle.

In C3 plants, the primary CO₂ fixation or carboxylation reaction involves the enzyme ribulose-1,5-diphosphate carboxylase, and the first stable product is a 3-carbon compound. C3 plants, such as hardwoods and conifers, are dominant in the temperate climate zones.

In C4 plants, an additional carboxylation reaction involving another enzyme, phosphoenol-pyruvate carboxylase, is the primary carboxylation reaction. The first stable carbon compound is a 4-carbon acid that is subsequently decarboxylated. The CO₂ thus released is refixed by the C3 cycle. Examples of C4 plants are tropical grasses, corn, and sugar cane.

Both C4 and C3 plants exhibit a range of ¹³C/¹²C isotopic ratios, but typical values are about −7 to about −13 per mil for C4 plants and about −19 to about −27 per mil for C3 plants (see, e.g., Stuiver et al., Radiocarbon 19:355 (1977)). Coal and petroleum fall generally in this latter range. The 13C measurement scale was originally defined by a zero set by Pee Dee Belemnite (PDB) limestone, where values are given in parts per thousand deviations from this material. The “δ13C” values are expressed in parts per thousand (per mil), abbreviated, ‰, and are calculated as follows:

δ¹3C(‰)=[(¹³C/¹²C) sample−(¹³C/¹²C) standard]/(¹³C/¹²C) standard×1000

Since the PDB reference material (RM) has been exhausted, a series of alternative RMs have been developed in cooperation with the IAEA, USGS, NIST, and other selected international isotope laboratories. Notations for the per mil deviations from PDB is δ¹³C. Measurements are made on CO₂ by high precision stable ratio mass spectrometry (IRMS) on molecular ions of masses 44, 45, and 46.

The compositions described herein include bioproducts produced by any of the methods described herein, including, for example, fatty esters and beta hydroxyl ester products. Specifically, the bioproduct can have a δ¹³C of about −28 or greater, about −27 or greater, −20 or greater, −18 or greater, −15 or greater, −13 or greater, −10 or greater, or −8 or greater. For example, the bioproduct can have a δ¹³C of about −30 to about −15, about −27 to about −19, about −25 to about −21, about −15 to about −5, about −13 to about −7, or about −13 to about −10. In other instances, the bioproduct can have a δ¹³C of about −10, −11, −12, or −12.3.

Bioproducts produced in accordance with the disclosure herein, can also be distinguished from petroleum based organic compounds by comparing the amount of ¹⁴C in each compound. Because ¹⁴C has a nuclear half-life of 5730 years, petroleum based fuels containing “older” carbon can be distinguished from bioproducts which contain “newer” carbon (see, e.g., Currie, “Source Apportionment of Atmospheric Particles”, Characterization of Environmental Particles, J. Buffle and H. P. van Leeuwen, Eds., 1 of Vol. I of the IUPAC Environmental Analytical Chemistry Series (Lewis Publishers, Inc.) 3-74, (1992)).

The basic assumption in radiocarbon dating is that the constancy of ¹⁴C concentration in the atmosphere leads to the constancy of ¹⁴C in living organisms. However, because of atmospheric nuclear testing since 1950 and the burning of fossil fuel since 1850, ¹⁴C has acquired a second, geochemical time characteristic. Its concentration in atmospheric CO₂, and hence in the living biosphere, approximately doubled at the peak of nuclear testing, in the mid-1960s. It has since been gradually returning to the steady-state cosmogenic (atmospheric) baseline isotope rate (¹⁴C/¹²C) of about 1.2×10-12, with an approximate relaxation “half-life” of 7-10 years. (This latter half-life must not be taken literally; rather, one must use the detailed atmospheric nuclear input/decay function to trace the variation of atmospheric and biospheric ¹⁴C since the onset of the nuclear age.)

It is this latter biospheric ¹⁴C time characteristic that holds out the promise of annual dating of recent biospheric carbon. ¹⁴C can be measured by accelerator mass spectrometry (AMS), with results given in units of “fraction of modern carbon” (fM). fM is defined by National Institute of Standards and Technology (NIST) Standard Reference Materials (SRMs) 4990B and 4990C. As used herein, “fraction of modern carbon” or “fM” has the same meaning as defined by National Institute of Standards and Technology (NIST) Standard Reference Materials (SRMs) 4990B and 4990C, known as oxalic acids standards HOxI and HOxII, respectively. The fundamental definition relates to 0.95 times the ¹⁴C/¹²C isotope ratio HOxI (referenced to AD 1950). This is roughly equivalent to decay-corrected pre-Industrial Revolution wood. For the current living biosphere (plant material), fM is approximately 1.1.

This is roughly equivalent to decay-corrected pre-Industrial Revolution wood. For the current living biosphere (plant material), fM is approximately 1.1.

The compositions described herein include bioproducts that can have an fM ¹⁴C of at least about 1. For example, the bioproduct of the invention can have an fM ¹⁴C of at least about 1.01, an fM ¹⁴C of about 1 to about 1.5, an fM ¹⁴C of about 1.04 to about 1.18, or an fM ¹⁴C of about 1.111 to about 1.124.

Another measurement of ¹⁴C is known as the percent of modern carbon (pMC). For an archaeologist or geologist using ¹⁴C dates, AD 1950 equals “zero years old”. This also represents 100 pMC. “Bomb carbon” in the atmosphere reached almost twice the normal level in 1963 at the peak of thermo-nuclear weapons. Its distribution within the atmosphere has been approximated since its appearance, showing values that are greater than 100 pMC for plants and animals living since AD 1950. It has gradually decreased over time with today's value being near 107.5 pMC. This means that a fresh biomass material, such as corn, would give a ¹⁴C signature near 107.5 pMC. Petroleum based compounds will have a pMC value of zero. Combining fossil carbon with present day carbon will result in a dilution of the present day pMC content. By presuming 107.5 pMC represents the ¹⁴C content of present day biomass materials and 0 pMC represents the ¹⁴C content of petroleum based products, the measured pMC value for that material will reflect the proportions of the two component types. For example, a material derived 100% from present day soybeans would give a radiocarbon signature near 107.5 pMC. If that material was diluted 50% with petroleum based products, it would give a radiocarbon signature of approximately 54 pMC.

A biologically based carbon content is derived by assigning “100%” equal to 107.5 pMC and “0%” equal to 0 pMC. For example, a sample measuring 99 pMC will give an equivalent biologically based carbon content of 93%. This value is referred to as the mean biologically based carbon result and assumes all the components within the analyzed material originated either from present day biological material or petroleum based material.

A bioproduct comprising one or more fatty esters as described herein can have a pMC of at least about 50, 60, 70, 75, 80, 85, 90, 95, 96, 97, 98, 99, or 100. In other instances, a fatty ester composition described herein can have a pMC of between about 50 and about 100; about 60 and about 100; about 70 and about 100; about 80 and about 100; about 85 and about 100; about 87 and about 98; or about 90 and about 95. In yet other instances, a fatty ester composition described herein can have a pMC of about 90, 91, 92, 93, 94, or 94.2.

### Utility of Fatty Ester Composition Compositions

Examples of fatty esters include fatty acid esters, such as those derived from short-chain alcohols, including fatty acid ethyl esters (“FAEE”) and fatty acid methyl esters (“FAME”), and those derived from long-chain fatty alcohols. The fatty esters and/or fatty ester compositions that are produced can be used, individually or in suitable combinations, as a biofuel (e.g., a biodiesel), an industrial chemical, or a component of, or feedstock for, a biofuel or an industrial chemical. In some aspects, the invention pertains to a method of producing a fatty ester composition comprising one or more fatty acid derivatives such as beta-hydroxy fatty acid esters, including, for example, beta-hydroxy FAEE, beta-hydroxy FAME and/or other beta-hydroxy fatty acid ester derivatives of longer-chain alcohols. In related aspects, the method comprises providing a genetically engineered production host suitable for making fatty esters and fatty ester compositions.

Accordingly, in one aspect, the invention features a method of making a fatty ester composition comprising a beta-hydroxy fatty ester. The method includes expressing in a host cell a gene encoding an ester synthase. In some embodiments, the gene encoding an ester synthase is selected from the enzymes classified as EC 2.3.1.75, and any other polypeptides capable of catalyzing the conversion of an acyl thioester to fatty esters, including, without limitation, ester synthases, acyl-CoA:alcohol transacylases, alcohol O-fatty acid-acyl-transferase, acyltransferases, and fatty acyl-coA:fatty alcohol acyltransferases, an engineered thioesterase or a suitable variant thereof. In other embodiments, the ester synthase gene is one that encodes wax/dgat, a bifunctional ester synthase/acyl-CoA: diacylglycerol acyltransferase from Simmondsia chinensis, Acinetobacter sp. ADP1, Alcanivorax borkumensis, Pseudomonas aeruginosa, Fundibacter jadensis, Arabidopsis thaliana, or Alkaligenes eutrophus. In some embodiments, the gene encoding an ester synthase is selected from the group consisting of: AtfA1 (an ester synthase derived from Alcanivorax borkumensis SK2, GenBank Accession No. YP.sub.-694462), AtfA2 (another ester synthase derived from Alcanivorax borkumensis SK2, GenBank Accession No. YP.sub.-693524), ES9 (an ester synthase from Marinobacter hydrocarbonoclasticus DSM 8798, GenBank Accession No. ABO21021), ES8 (another ester synthase derived from Marinobacter hydrocarbonoclasticus DSM 8798, GenBank Accession No. ABO21020), and variants thereof. In a particular embodiment, the gene encoding the ester synthase or a suitable variant is overexpressed.

In another aspect, the invention features a method of making a fatty acid derivative, for example, a fatty ester, the method comprising expressing in a host cell a gene encoding an ester synthase polypeptide comprising the amino acid sequence of SEQ ID NO:18, 24, 25, or 26, or a variant thereof. In certain embodiments, the polypeptide has ester synthase and/or acyltransferase activity. In some embodiments, the polypeptide has the capacity to catalyse the conversion of a thioester to a fatty acid and/or a fatty acid derivative such as a fatty ester. In a particular embodiment, the polypeptide has the capacity to catalyze the conversion of a fatty acyl-CoA and/or a fatty acyl-ACP to a fatty acid and/or a fatty acid derivative such as a fatty ester, using an alcohol as substrate. In alternative embodiments, the polypeptide has the capacity to catalyze the conversion of a free fatty acid to a fatty ester, using an alcohol as substrate.

In certain embodiments, an endogenous thioesterase of the host cell, if present, is unmodified. In certain other embodiments, the host cell expresses an attenuated level of a thioesterase activity or the thioesterase is functionally deleted. In some embodiments, the host cell has no detectable thioesterase activity. As used herein the term “detectable” means capable of having an existence or presence ascertained. For example, production of a product from a reactant (e.g., production of a certain type of fatty acid esters) is desirably detectable using the methods provided herein. In certain embodiments, the host cell expresses an attenuated level of a fatty acid degradation enzyme, such as, for example, an acyl-CoA synthase, or the fatty acid degradation enzyme is functionally deleted. In some embodiments, the host cell has no detectable fatty acid degradation enzyme activity. In particular embodiments, the host cell expresses an attenuated level of a thioesterease, a fatty acid degradation enzyme, or both. In other embodiments, the thioesterase, the fatty acid degradation enzyme, or both, are functionally deleted. In some embodiments, the host cell has no detectable thioesterase activity, acyl-CoA synthase activity, or neither. In some embodiments, the host cell can convert an acyl-ACP or acyl-CoA into fatty acids and/or derivatives thereof such as esters, in the absence of a thioesterase, a fatty acid derivative enzyme, or both. Alternatively, the host cell can convert a free fatty acid to a fatty ester in the absence of a thioesterase, a fatty acid derivative enzyme, or both. In certain embodiments, the method further includes isolating the fatty acids or derivatives thereof from the host cell.

In certain embodiments, the fatty acid derivative is a fatty ester. In certain embodiments, the fatty acid or fatty acid derivative is derived from a suitable alcohol substrate such as a short- or long-chain alcohol. In some embodiments, the fatty acid or fatty acid derivative is present in the extracellular environment. In certain embodiments, the fatty acid or fatty acid derivative is isolated from the extracellular environment of the host cell. In some embodiments, the fatty acid or fatty acid derivative is spontaneously secreted, partially or completely, from the host cell. In alternative embodiments, the fatty acid or derivative is transported into the extracellular environment, optionally with the aid of one or more transport proteins. In other embodiments, the fatty acid or fatty acid derivative is passively transported into the extracellular environment.

In another aspect, the invention features an in vitro method of producing a fatty acid and/or a fatty acid derivative extracellulary comprising providing a substrate and a purified ester synthase comprising the amino acid sequence of SEQ ID NO:18, 24, 25, or 26, or a variant thereof. In some embodiments, the method comprising culturing a host cell under conditions that allow expression or overexpression of an ester synthase polypeptide or a variant thereof, and isolating the ester synthase from the cell. In some embodiments, the method further comprising contacting a suitable substrate such with the cell-free extract under conditions that permit production of a fatty acid and/or a fatty acid derivative.

In some embodiments, the ester synthase polypeptide comprises the amino acid sequence of SEQ ID NO:18, 24, 25, or 26, with one or more amino acid substitutions, additions, insertions, or deletions, and the polypeptide has ester synthase and/or acyltransferase activity. In certain embodiments, the ester synthase polypeptide has increased ester synthase and/or transferase activity. For example, the ester synthase polypeptide is capable, or has an improved capacity, of catalyzing the conversion of thioesters, for example, fatty acyl-CoAs or fatty acyl-ACPs, to fatty acids and/or fatty acid derivatives. In particular embodiments, the ester synthase polypeptide is capable, or has an improved capacity, of catalyzing the conversion of thioester substrates to fatty acids and/or derivatives thereof, such as fatty esters, in the absence of a thioesterase activity, a fatty acid degradation enzyme activity, or both. For example, the polypeptide converts fatty acyl-ACP and/or fatty acyl-CoA into fatty esters in vivo, in the absence of a thioesterase or an acyl-CoA synthase activity. In alternative embodiments, the polypeptide is capable of catalyzing the conversion of a free fatty acid to a fatty ester, in the absence of a thioesterase activity, a fatty acid degradation enzyme activity, or both. For example, the polypeptide can convert a free fatty acid into a fatty ester in vivo or in vitro, in the absence of a thioesterase activity, an acyl-CoA synthase activity, or both.

In some embodiments, the ester synthase polypeptide is a variant comprising the amino acid sequence of SEQ ID NO:18, 24, 25, or 26, with one or more non-conserved amino acid substitutions, wherein the ester synthase polypeptide has ester synthase and/or acyltransferase activity. In certain embodiments, the ester synthase polypeptide has improved ester synthase and/or acyltransferase activity. For example, a glycine residue at position 395 of SEQ ID NO:18 can be substituted with a basic amino acid residue, such that the resulting ester synthase variant retains or has improved ester synthase and/or acyltransferase activity. In an exemplary embodiment, the glycine residue at position 395 of SEQ ID NO:18 is substituted with an arginine or a lysine residue, wherein the resulting ester synthase variant retains or has improved capacity to catalyze the conversion of a thioester into a fatty acid and/or a fatty acid derivative such as a fatty ester.

In some embodiments, the ester synthase variant comprises one or more of the following conserved amino acid substitutions: replacement of an aliphatic amino acid, such as alanine, valine, leucine, and isoleucine, with another aliphatic amino acid; replacement of a serine with a threonine; replacement of a threonine with a serine; replacement of an acidic residue, such as aspartic acid and glutamic acid, with another acidic residue; replacement of residue bearing an amide group; exchange of a basic residue, such as lysine and arginine, with another basic residue; and replacement of an aromatic residue, such as phenylalanine and tyrosine, with another aromatic residue. In some embodiments, the ester synthase variant has about 1, 2, 3, 4, 5, 6, 7, 8, 9, 10, 15, 20, 30, 40, 50, 60, 70, 80, 90, 100, or more amino acid substitutions, additions, insertions, or deletions. In some embodiments, the polypeptide variant has ester synthase and/or acyltransferase activity. For example, the ester synthase polypeptide is capable of catalyzing the conversion of thioesters to fatty acids and/or fatty acid derivatives, using alcohols as substrates. In a non-limiting example, the polypeptide is capable of catalyzing the conversion of a fatty acyl-CoA and/or a fatty acyl-ACP to a fatty acid and/or a fatty acid ester, using a suitable alcohol substrate, such as, for instance, a methanol, ethanol, propanol, butanol, pentanol, hexanol, heptanol, octanol, decanol, dodecanol, tetradecanol, or hexadecanol. In another non-limiting example, the ester synthase polypeptide is capable of catalyzing the conversion of a fatty acyl-ACP and/or a fatty acyl-CoA to a fatty acid and/or a fatty acid ester, in the absence of a thioesterase, a fatty acid degradation enzyme, or both. In a further embodiment, the polypeptide is capable of catalyzing the conversion of a free fatty acid into a fatty ester in the absence of a thioesterase, a fatty acid degradation enzyme, or both.

The invention is further illustrated by the following examples. The examples are provided for illustrative purposes only. They are not to be construed as limiting the scope or content of the invention in any way.

### EXAMPLES

### Example 1

### Production of E. coli MG1655DAM1/pDS57

An ester synthase gene encoding an ester synthase ES9 from Marinobacter hydrocarbonoclasticus DSM8789 gene (GenBank Accession No. ABO21021: SEQ ID NO:1) was synthesized by DNA2.0 (Menlo Park, Calif.) and used to construct plasmid pDS57 (SEQ ID NO:3). The synthesized gene was then cloned into a pCOLADuet-1 plasmid (EMD Chemicals, Inc., Gibbstown, N.J.) to form a pHZ1.97-ES9 construct. The internal BspHI restriction site of the ester synthase gene was then removed by site-directed mutagenesis, using the QuikChange™ Multi Kit (Stratagene, Carlsbad, Calif.) and the primer:

(SEQ ID NO: 4)
ES9BspF: 5′-CCCAGATCAGTTTTATGATTGCCTCGCTGG-3′

This primer introduced a silent mutation into the ester synthase gene. The resulting plasmid was called pDS32. pDS32 was then used as a template to amplify the ester synthase gene using the following primers:

ES9BspH-Forward:
(SEQ ID NO: 5)
5′-ATCATGAAACGTCTCGGAAC-3′
ES9Xho-Reverse:
(SEQ ID NO: 6)
5′-CCTCGAGTTACTTGCGGGTTCGGGCGCG-3′

The PCR product was subject to restriction digestions with BspHI and XhoI. This digestion fragment was then ligated into a pDS23 plasmid (as described below) that had been digested with NcoI and XhoI, to form a plasmid pDS33.ES9 (SEQ ID NO:7).

Construction of pDS23

A Pspc promoter (SEQ ID NO:8) was obtained by PCR amplification, using Phusion™ Polymerase (New England Biolabs, Inc., Ipswich, Mass.) from E. coli MG1655 chromosomal DNA. The following primers were used:

PspcIFF:
(SEQ ID NO: 9)
5′-AAAGGATGTCGCAAACGCTGTTTCAGTACACTCTCTCAATAC-3′
PspcIFR:
(SEQ ID NO: 10)
5′-GAGCTCGGATCCATGGTTTAGTGCTCCGCTAATG-3′

The PCR fragment was then used to replace the lacI_{q} and Ptrc promoter sequences of a plasmid OP-80 (SEQ ID NO:11), which was constructed as described below.

### Construction of Plasmid OP-80.

A commercial vector pCL1920 (see, Lerner, et al., Nucleic Acids Res. 18:4631 (1990)), carrying a strong transcriptional promoter, was used as the starting point. The pCL1920 vector was digested with AflII and sfoI (New England Biolabs, Ipswich, Mass.). Three DNA fragments were produced, among which, a 3737-bp fragment was gel-purified using a gel-purification kit (Qiagen, Inc., Valencia, Calif.).

In parallel, a DNA fragment comprising the Ptrc promoter and the lacI sequences was obtained from a plasmid pTrcHis2 (Invitrogen, Carlsbad, Calif.) using the following primers:

(SEQ ID NO: 12)
LF302: 5′-ATATGACGTCGGCATCCGCTTACAGACA-3′
(SEQ ID NO: 13)
LF303: 5′-AATTCTTAAGTCAGGAGAGCGTTCACCGACAA-3′

These primers also introduced the restriction sites for ZraI and AflII. The PCR product was purified using a PCR-purification kit (Qiagen, Inc., Valencia, Calif.) and digested with ZraI and AflII. The digestion product was gel-purified and ligated with the 3737-bp fragment (described above). The ligation mixture was then transformed into TOP100 chemically competent cells (Invitrogen, Carlsbad, Calif.). The transformants were selected on Luria agar plates containing 100 μg/mL spectinomycin during overnight incubation. Resistant colonies were identified, and plasmids within these colonies were purified, and verified with restriction digestion and sequencing. One plasmid produced this way was retained, and given the name of OP-80 (SEQ ID NO:11).

The PCR fragment comprising the Pspc promoter (described above) was cloned into the BseRI and NcoI restriction sites of OP-80 using the InFusion™ Cloning Kit (Clontech, Menlo Park, Calif.). The resulting plasmid was given the name pDS22. pDS22 still possessed a lacZgene sequence downstream of the multiple cloning site. The lacZsequence was removed with PCR employing the following primers:

| (SEQ ID NO: 14) | |
|---|---|
| pCLlacDF: | 5′-GAATTCCACCCGCTGACGAGCTTA-3′ |
| (SEQ ID NO: 15) | |
| pCLEcoR: | 5′ -CGAATTCCCATATGGTACCAG-3′ |

The PCR product was subject to restriction digestion by EcoRI. The digested product was subsequently self-ligated to form a plasmid named pDS23, which did not contain lacI_{q}, lacZ or promoter Ptrc sequence.

The plasmid pDS33.ES9 (SEQ ID NO:7; described above) was again digested with BspHI and XhoI. After digestion, the fragment was ligated with an OP-80 plasmid ((SEQ ID NO:11; described above) that had been previously linearized using NcoI/XhoI restriction digestions.

The ligation product was transformed into TOP10® One Shot chemically competent cells (Invitrogen, Carlsbad, Calif.). Cells were then plated on LB plates containing 100 μg/mL spectinomycin, and incubated overnight at 37° C. After overnight growth, several colonies were purified and the sequence of the inserts verified. The plasmid was given the name pDS57 (SEQ ID NO:3).

E. coli DAM1 strain was made electrocompetent using standard methods. The competent cells were then transformed with plasmid pDS57 and plated on LB plates containing 100 μg/mL of spectinomycin, and incubated overnight at 37° C. Resistant colonies were purified and the presence of the pDS57 plasmid was confirmed using restriction digestion and sequencing. The resulting construct was given the name E. coli DAM1/pDS57.

### Example 2

### Production of a Fatty Ester Composition Comprising Beta-Hydroxy Fatty Acid Esters by DG5 pDS57 and DIR1 pDS57

This example describes processes used to produce a fatty ester composition using a genetically modified E. coli strains DG5 pDS57 and DIR1 pDS57, overexpressing an ester synthase from Marinobacter hydrocarbonoclasticus. These strains contain only one heterologous polynucleotide sequence encoding an ester synthase.

A fermentation and recovery process was used to produce biodiesel of commercial grade quality by fermentation of carbohydrates. The fermentation process produced a mix of fatty acid methyl esters (FAME), including beta-hydroxy methyl ester and fatty acid ethyl esters (FAEE) for use as a biodiesel using the genetically engineered microorganisms described herein.

### Fermentation

The following details correspond to the process run in a 5 L laboratory fermentor. Cells from a frozen stock were grown in LB media for a few hours and then transferred to a flask containing a minimal media consisting of: 30 g/L glucose, 100 mM bis-tri buffer at pH 7.0, 3.0 g/L of KH₂PO₄, 6.0 g/L Na₂HPO₄, 2.0 g/L of NH₄Cl, 0.24 g/L of MgSO₄.7H₂O, 0.034 g/L of ferric citrate, 0.12 ml/L of 1M HCl, 0.02 g/L of ZnCl₂.4H₂O, 0.02 g/L of CaCl₂.2H₂O, 0.02 g/L of Na₂MoO₄.2H₂O, 0.019 g/L CuSO₄5H₂O, 0.005 g/L H₃BO₃ and 1 mg/L of thiamine. The shake flask was incubated overnight at 32° C., and 200 rpm. 50 ml/L aliquots of the overnight cultures were used to inoculate the fermentation tanks.

The media in the tanks had the following composition: 5 g/L glucose, 4.89 g/L of KH₂PO₄, 0.5 g/L of (NH₄)₂SO₄, 0.15 g/L of MgSO₄.7H₂O, 2.5 g/L Bactocasaminoacids, 0.034 g/L of ferric citrate, 0.12 ml/L of 1M HCl, 0.02 g/L of ZnCl₂.4H₂O, 0.02 g/L of CaCl₂.2H₂O, 0.02 g/L of Na₂MoO₄.2H₂O, 0.019 g/L CuSO4.5H2O, 0.005 g/L H₃BO₃ and 1.25 ml/L of a vitamin solution. The vitamin solution contained: 0.06 g/l riboflavin, 5.40 g/L pantothenic acid, 6.0 g/L niacin, 1.4 g/L pyridoxine and 0.01 g/L folic acid. The preferred conditions for the fermentation were 32° C., pH 6.8 and dissolved oxygen (DO) equal to 25% of saturation. pH was maintained by addition of NH₄OH, which also acts as nitrogen source for cell growth. When the initial 5 g/L of glucose was almost consumed, a feed consisting of about 600 g/L glucose, 1.6 g/L KH₂PO₄, 3.9 g/L MgSO₄.7H₂O, 0.13 g/L ferric citrate and 30 ml/L of methanol was supplied to the fermentor. The feed rate was set up to match the cells growth rate and avoid accumulation of glucose. By avoiding glucose accumulation, it was possible to reduce or eliminate the formation of by-products such as acetate, formate and ethanol, which are commonly produced by E. coli. In the early phases of the growth, the production of FAME was induced by the addition of 1 mM IPTG and 20 ml/L of pure methanol. After most of the cell growth was complete, the feed rate was maintained at a rate of up to 10 g glucose/L/h. The fermentation was continued for a period of 3 days.

For production of FAEE, fermentation was performed as described above except that pure ethanol was substituted for methanol.

FAME and FAEE production rates reached their peak when the cells decreased their growth rate and started approaching stationary phase. FAME titers between 5 and 10 g/L and FAEE titers between 16 and 30 g/L were routinely obtained using this protocol with these strains.

Following fermentation, the fatty ester composition was separated from the fermentation broth using any suitable recovery method, including various methods well known in the art. The recovered ester composition was further subjected to optional polishing steps, including polishing steps known in the art. An exemplary recovery method and polishing step are described below.

### Example 3

### Production of Biodiesel by Fermentation using DAM1 pDS57

This example demonstrates processes used to produce a fatty ester composition with DAM1 pDS57. A fermentation and recovery process was used to produce biodiesel of commercial grade by fermentation of carbohydrates at the 5 liter scale using the process described above for DG5 pDS57 and DIR1 pDS57. The fermentation process produced a mix of fatty acid methyl esters (FAME), including beta-hydroxy methyl ester and fatty acid ethyl esters (FAEE) at a level of up to 8 g/L.

### Scale-Up of Biodiesel Production by Fermentation

This example demonstrates production of a fatty ester composition using genetically modified microorganisms and processes to similar to those described above. A fermentation and recovery process was used to produce biodiesel of commercial grade by fermentation of carbohydrates. The fermentation process produced a mix of fatty acid methyl esters (FAME), including beta-hydroxy methyl ester and fatty acid ethyl esters (FAEE) useful as biodiesel.

### Fermentation

The fermentation process described herein was carried out by using methods well known to those of ordinary skill in the art. For example, the fermentation process can be carried out in a 2 to 5 L lab-scale fermentor, as described above for DAM1 pDS57. Alternatively, the fermentation process can be scaled up using the methods described herein or alternative methods known in the art.

The following details correspond to the process when run in a 750 L pilot plant fermentor. Cells from a frozen stock were grown in LB media for a few hours and then transferred to a fermentor with defined media consisting of: 30 g/L glucose, 2.0 g/L of KH₂PO₄, 0.15 g/L of (NH₄)₂SO₄, 0.5 g/L of MgSO₄.7H₂O, 5 g/L Bactocasaminoacids, 0.034 g/L of ferric citrate, 0.12 ml/L of 1MHCl, 0.02 g/L of ZnCl₂.4H₂O, 0.02 g/L of CaCl₂.2H₂O, 0.02 g/L of Na₂MoO₄.2H₂O, 0.019 g/L CuSO4.5H2O, 0.005 g/L H₃BO₃ and 1.25 ml/L of a vitamin solution. This solution contained: 0.06 g/L Riboflavina, 5.40 g/L pantothenic acid, 6.0 g/L niacin, 1.4 g/L piridoxine and 0.01 g/L folic acid. The preferred conditions for the fermentation were 32° C., pH 6.8 and dissolved oxygen (DO) equal to 25% of saturation. The pH was maintained by addition of NH₄OH, which also acts as nitrogen source for cell growth. This fermentor allows the propagation of cells to a reasonable density, after which they are used to inoculate the pilot plant tank.

The pilot plant tank contains the same medium as the inoculum fermentor described above, but with only 5 g/L of glucose. When the initial 5 g/L of glucose was almost consumed, a feed consisting of about 600 g/L glucose, 1.6 g/L of KH₂PO₄, 3.9 g/L MgSO₄.7H₂O, 0.13 g/L ferric citrate and 30 ml/L of methanol was supplied to the fermentor. The feed rate was set up to match the cell growth rate and avoid accumulation of glucose in the fermentor. By avoiding glucose accumulation, it was possible to reduce or eliminate the formation of by-products such as acetate, formate and ethanol, which are commonly produced by E. coli. In the early phases of growth, the production of FAME was induced by the addition of 1 mM IPTG and 20 ml/L of pure methanol. After most of the cell growth was complete, the feed rate was maintained at a rate of up to 10 g glucose/L/h. The fermentation was continued for a period of 3 days.

FAME production rate reached its peak when the cells decreased their growth rate and started approaching stationary phase. FAME titers between 45 and 55 g/L were routinely obtained using this protocol, with concentrations of beta-hydroxy (“B—OH”) fatty acid methyl esters (“FAMEs”) from 2 to 8 g/L.

### Example 4

### Identification of Beta-Hydroxy Esters in Fermentation Broth

The samples were derivatized with BSTFA for free fatty acid analysis. Samples containing derivatized FAME or FAEE were analyzed by gas chromatography mass spectroscopy (GC-MS) and/or by gas chromatography with a flame ionization detector (GC-FID) (See US Patent Publication 20100257777). These analyses allowed detection of presence of beta-hydroxy (3-OH) esters in the samples.

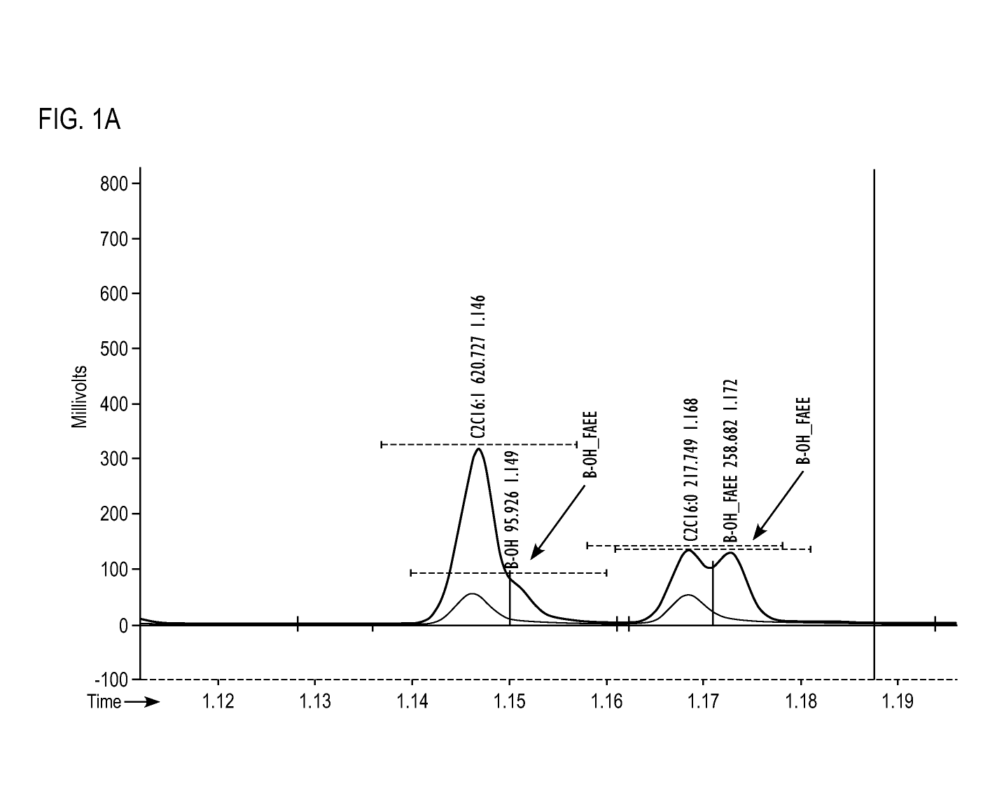

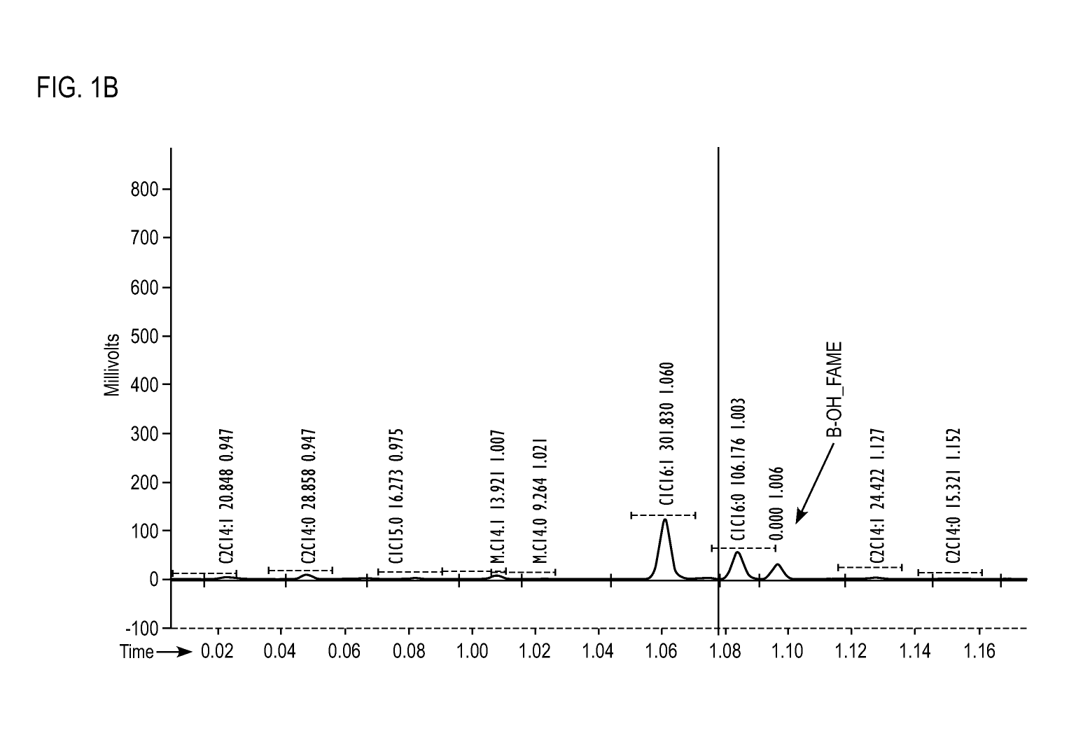

For derivatized FAEE samples, peaks split on GC-FID, whereas results of GC-FID analysis for derivatized FAME samples showed clearly separated peaks (FIG. 1).

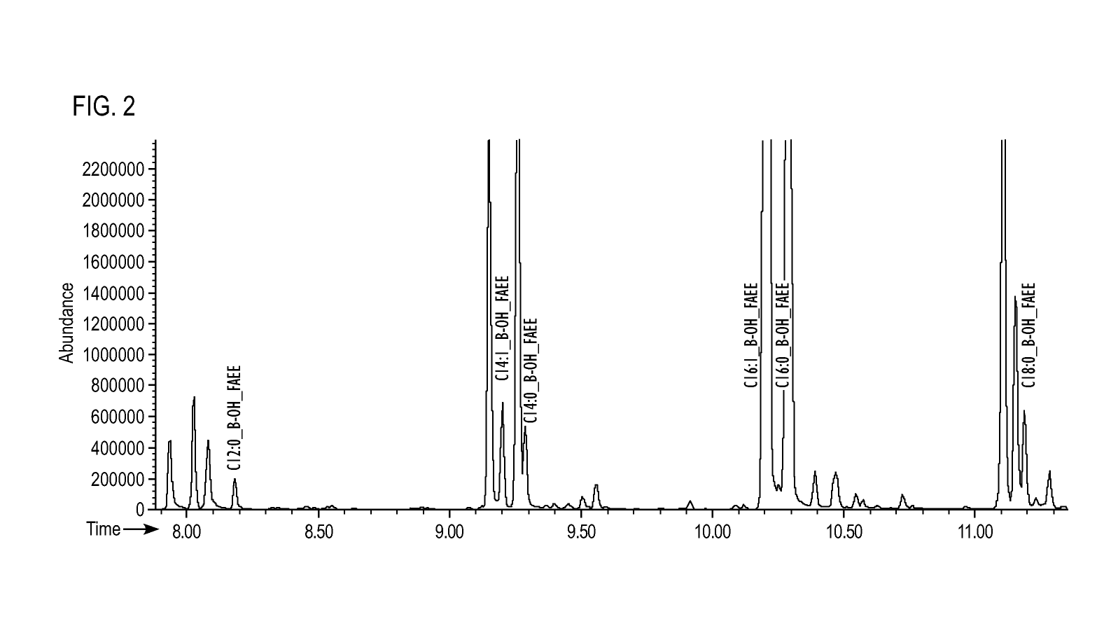

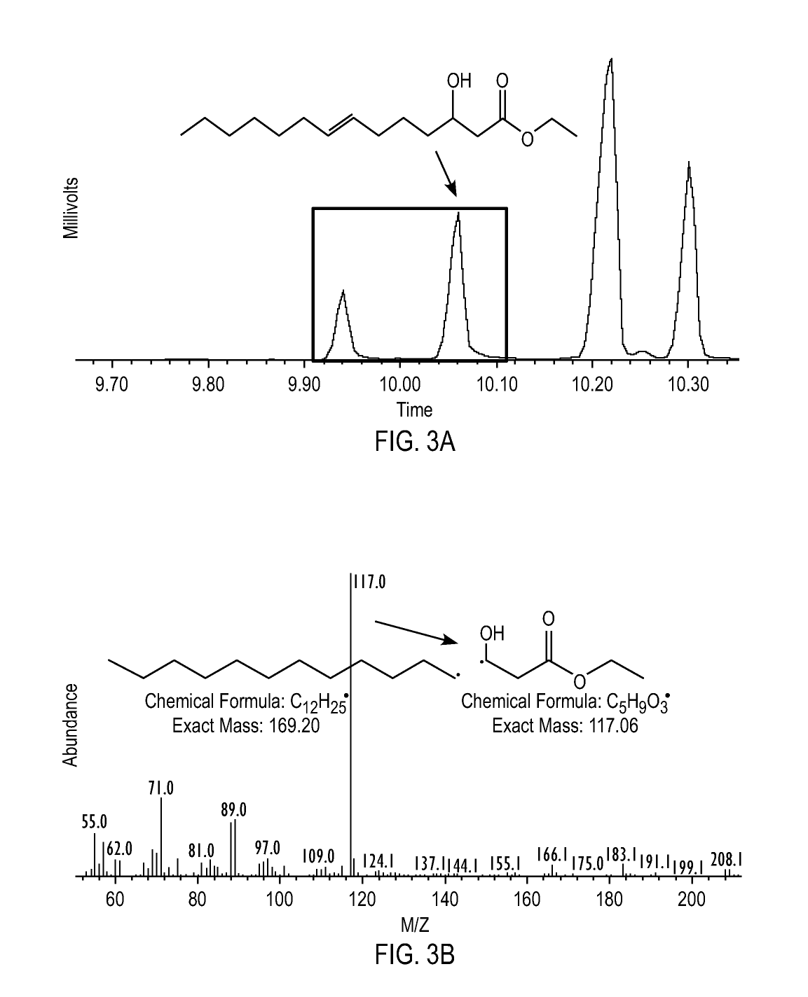

Samples containing derivatized FAEE were run on GC-MS; the left portion of the peak shows the presence of beta-hydroxy esters and the right half of the peak includes non-hydroxylated esters. (FIG. 2 and FIG. 3).

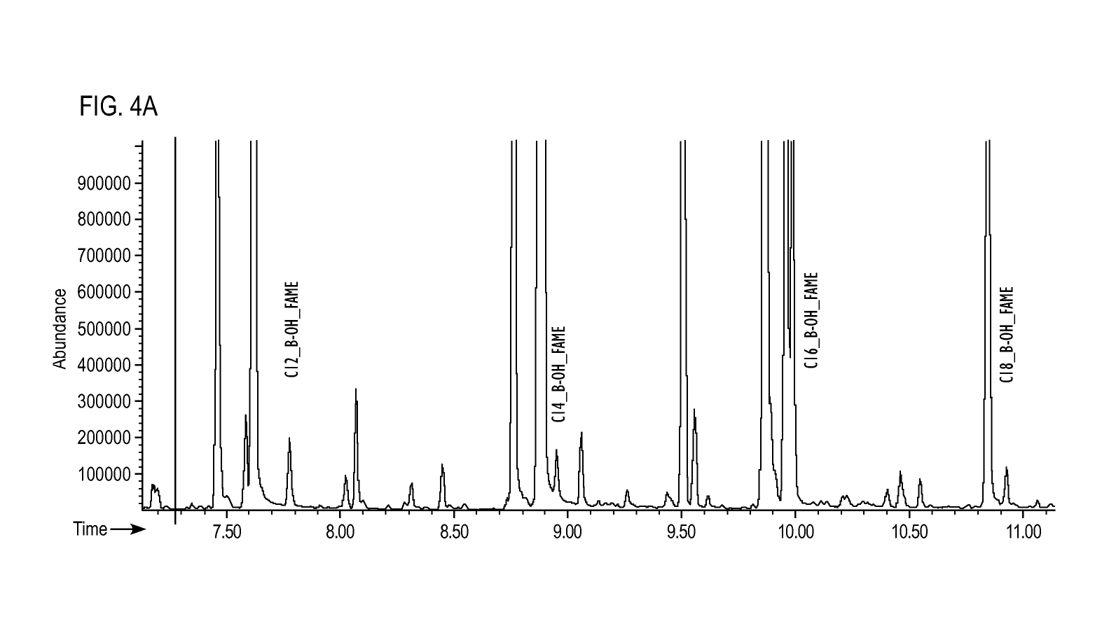

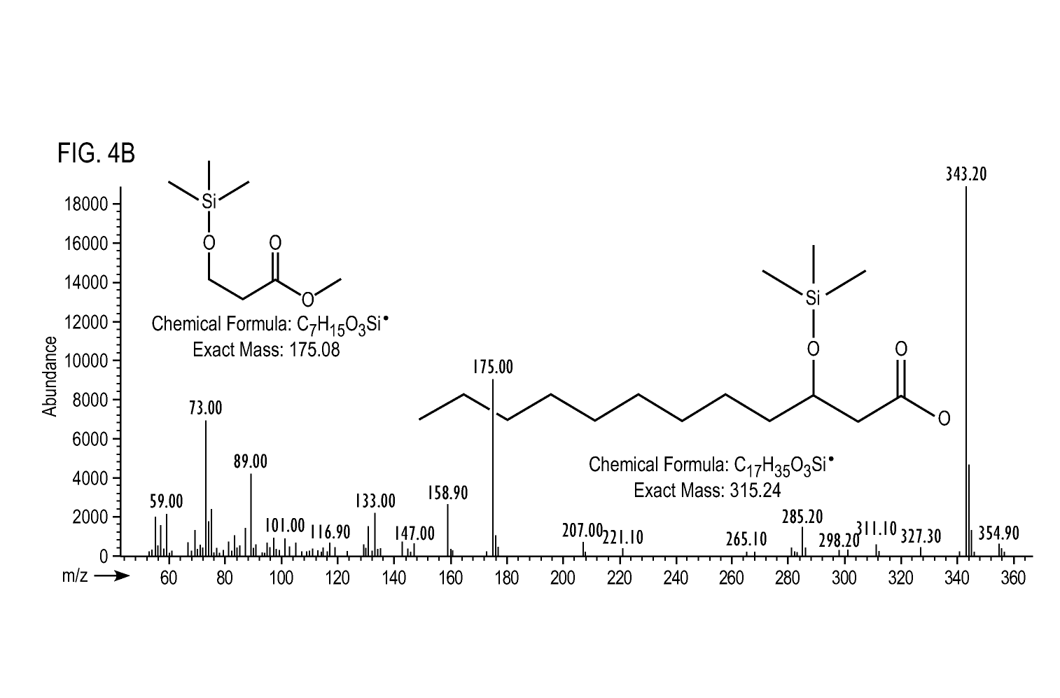

Underivitized FAEE samples were run on GC-FID and GC-MS. All hydroxy esters co-eluted with the corresponding non-hydroxy esters on GC-MS (FIG. 4) and split on GC-FID (FIG. 1) separate out on both the instruments. See chromatograms below (FIGS. 3A and B).

For FAME samples, peaks separate on the GC-FID (FIG. 1) and also GC-MS for both derivitized and underivitized FAME, with the only difference being a shift of the peaks towards right for derivatized samples). These peaks were identified on GC-MS as hydroxy compound. See FIGS. 4A and B.

Structural elucidation of all the hydroxy compounds (derivitized and underivitized C12, C14, C16 and C18 FAME and FAEE) was done on chemdraw software to determine the exact masses of each of the FAEE and FAME beta-hydroxy compounds and the corresponding fragment ions. The ions were extracted by single ion monitoring on GC-MS and the presence of the beta-hydroxy compounds was thereby confirmed.

A summary of the data for the various tested strains is provided in Table 1, below. Those strains that produced beta-hydroxy esters are indicated with an “X” under the column “B—OH Esters”.

TABLE 1
Summary of Beta-Hydroxy Ester Production by Recombinant Host Cells.

| | 1-enzyme | 3-enzyme | | B-OH | |
|---|---|---|---|---|---|
| Strains | pathway | pathway | pDS57 | Esters | Comments |
| DG5 pDS57 | X | | X | X | |
| DG5 pDS57 (G), DG5 pDS57 (I) | X | | X | X | |
| DV2 trc_tesA_fadD pDS57 (A), | | X | X | X | |
| DG5 trc_tesA_fadD pDS57 (I), | | | | | |
| DV2 trc_tesA_fadD | | X | | | |
| DV2 trc_tesA_fadD pDS57 | | X | X | X | |
| DG5 trc_tesA_fadD pDS57 | | X | X | X | |
| IDV2 | | X | | | Pilot plant- ester |
| | | | | | synthase aftA1 |
| IDV2 | | X | | | Pilot plant- ester |
| | | | | | synthase aftA1 |

All the samples containing fatty acids ethyl esters were analyzed for beta-hydroxy compounds by analyzed by GC-FID. The total titer for C14 beta-hydroxy compound from an exemplary run was found to be 2-6 g-L giving a total estimate of 15-20% of the total FAEE in the sample. For the samples with fatty acids methyl esters, the peaks were separate. One of the samples with highest titer was taken, run on GC-MS and a rough estimate was done based on the assumption that the peak area ratio of FAEE/OH-FAEE and peak area ratio of FAME/OH-FAME is the same. The total estimate of BETA-HYDROXY FAME in the sample was found to be 6-8% of the total titer of FAME.

As can be seen from Table 1, beta-hydroxy esters were produced under all conditions when the ester synthase ES9 was present, for strains having either the one enzyme or three enzyme pathway (FIG. 5) and in the presence of methanol or ethanol. No beta-hydroxy esters were observed in strains having TesA or with atfA1 in the absence of ester synthase ES9.

### Fatty Ester Compositions

In certain instances, the genetically modified strains of E. coli described herein when fermented, recovered, and/or polished as described herein produced a mixture of FAME with the composition profile shown in Table 2.

TABLE 2
Fatty Acid Ester Composition

| Componenet | Percentage |
|---|---|
| Methyl octanoate (C8:0) | 0-5% |
| Methyl decanoate (C10:0) | 0-2% |
| Methyl dodecanoate (C12:0): | 0-5% |
| Methyl dodecenoate (C12:1): | 0-10% |
| Methyl tetradecanoate (C14:0): | 30-50% |
| Methyl 7-tetradecenoate (C14:1): | 0-10% |
| Methyl hexadecanoate (C16:0): | 0-15% |
| Methyl 9-hexadecenoate (C16:1): | 10-40% |
| Methyl 11-octadecenoate (C18:1): | 0-15% |

Of the total FAMEs, from 5 to 25% are the corresponding beta-hydroxy forms of the methyl esters. The actual composition of the FAME mixture is dependent on the specific E. coli strain used for production, as strains with different genetic mutations may be used to improve production, but not on the conditions of the fermentation or recovery processes. In other words, the percentages will be in the ranges described, however, the exact distribution will depend on the strain, for example, oil from DG5 PDS57 is different than oil from DAM1 pDS57. Accordingly, the lots of biodiesel produced from a given E. coli strain were consistent from batch to batch. The percentage of each of the various methyl esters, e.g. a percentage of methyl octanoate (C8:0) of 0-5%, is expressed as a percentage of total fatty esters.

In one example of the process described above, the composition of the biodiesel in the fermentation broth is shown in Table 3.

TABLE 3
Fatty Acid Ester Composition (DG5 pDS57).

| Component | Percentage |
|---|---|
| Methyl octanoate (C8:0) | 2.1% |
| Methyl decanoate (C10:0) | 0.9% |
| Methyl dodecanoate (C12:0): | 9.6% |
| Methyl dodecenoate (C12:1): | 4.3% |
| Methyl tetradecanoate (C14:0): | 36.3% |
| Methyl 7-tetradecenoate (C14:1): | 9.4% |
| Methyl hexadecanoate (C16:0): | 9.7% |
| Methyl 9-hexadecenoate (C16:1): | 23.7% |
| Methyl 11-octadecenoate (C18:1): | 3.5% |

12.8% of the total fatty acid methyl esters were beta-hydroxy methyl esters. In another example of the process described above, the composition of the biodiesel was in the fermentation broth was as follows: fermentation broth is shown in Table 4.

TABLE 4
Fatty Acid Ester Composition (DAM1 pDS57).

| Component | Percentage |
|---|---|
| Methyl octanoate (C8:0) | 1.6% |
| Methyl decanoate (C10:0) | 0.6% |
| Methyl dodecanoate (C12:0): | 8.5% |
| Methyl dodecenoate (C12:1): | 4.3% |
| Methyl tetradecanoate (C14:0): | 36.6% |
| Methyl 7-tetradecenoate (C14:1): | 8.4% |
| Methyl hexadecanoate (C16:0): | 8.6% |
| Methyl 9-hexadecenoate (C16:1): | 26.7% |
| Methyl 11-octadecenoate (C18:1): | 4.7% |

12.0% of the total fatty acid methyl esters were beta-hydroxy methyl esters.

The results obtained are acceptable under the defined set of biodiesel characteristics determined through standardized ASTM tests. These tests, their nomenclature and allowed limits are described in the ASTM Standards D 6751, which are summarized in Table 5, below.

TABLE 5
Specification for Biodiesel (B100).

| | ASTM | | | |
|---|---|---|---|---|
| Property | Method | Limits | | Units |
| Calcium and Magnesium, | EN 14538 | 5. | max | Ppm (μg/g) |
| combined | | | | |
| Flash Point (closed cup) | D93 | 93.0 | | ° C. |
| Alcohol Control (one of | | | | |
| the following must be met) | | | | |
| 1. Methanol Content | EN 14110 | 0.2 | max | % volume |
| 2. Flash Point | D93 | 130 | min | ° C. |
| Water & Sediment | D2709 | 0.050 | max | % volume |
| Kinematic Viscosity, | D445 | 1.9-6.0 | | mm²/sec |
| 40° C. | | | | |
| Sulfated Ash | D874 | 0.020 | max | % mass |
| Sulfur S15 Grade | D5453 | 0.0015 | max | % mass (ppm) |
| Sulfur S500 Grade | D5453 | 0.05 | max | % mass (ppm) |
| Copper Strip Corrosion | D130 | No. 3 | max | |
| Cetane Number | D613 | 47 | min | |
| Cloud Point | D2500 | Report to | | ° C. |
| | | customer | | |
| Carbon Residue | D4530 | 0.050 | max | % mass |
| 100% sample^{a} | | | | |
| Acid Number | D664 | 0.50 | max | mg KOH/gm |
| Free Glycerin | D6584 | 0.020 | max | % mass |
| Total Glycerin | D6584 | 0.240 | max | % mass |
| Phosphorus Content | D 4951 | 0.001 | max | % mass |
| Distillation, T90 AET | D 1160 | 360 | max | ° C. |
| Sodium/Potassium, | EN 14538 | 5 | max | ppm |
| combined | | | | |
| Oxidation Stability | EN 14112 | 3 | min | hours |
| Cold Soak Filterability | Annex to | 360 | max | seconds |
| | D6751 | | | |
| For use in temperatures | Annex to | 200 | max | seconds |
| below −12° C. | D6751 | | | |

### Source: National Renewable Energy Laboratory, Biodiesel Handling and Use Guide, Fourth Edition, NREL/TP-540-43672, January 2009.

The impurity profile of the fatty ester composition produced using the genetically modified microorganism described above for scale-up of biodiesel production by fermentation. After isolation of the fatty ester composition after two centrifugations, the fatty ester composition was subjected to analysis. The results of the analysis are set forth in Table 6, below. The test methods followed the protocols set out in the ASTM D 6571 biodiesel standard.

TABLE 6
ASTM D 6571 Biodiesel Standard.

| Component | Test Method | Results | |
|---|---|---|---|
| Sulfur | D 5453 | 23 | ppm |
| Sulfated Ash | D 874 | <0.001 | |
| Microcarbon Residue | D 4530 | 0.07 | wt. % |
| Water and Sediment | D 2709 | 0.01 | vol. % |
| Sodium | EN 14538 | 2.3 | ppm |
| Potassium | EN 14538 | <0.1 | ppm |
| Magnesium | EN 14538 | <0.1 | ppm |
| Calcium | EN 14538 | 0.8 | ppm |
| Methanol content | EN 14110 | 0.03 | vol. % |
| Phosphorous | D 4951 | <0.0001 | wt. % |

1. A recombinant microorganism comprising
a heterologous polynucleotide sequence encoding a polypeptide having an ester synthase (EC 2.3.1.75) activity, wherein the recombinant microorganism produces an ester composition comprising a beta-hydroxy fatty ester in the presence of a carbon source.

2. The recombinant microorganism of claim 1, further comprising a heterologous polynucleotide sequence encoding a polypeptide having a thioesterase (EC 3.1.2.14 or EC 3.1.1.5) and an acyl-CoA synthase (EC 2.3.1.86) activity.

3. A method of producing a fatty ester composition comprising beta-hydroxy fatty esters, the method comprising culturing the recombinant microorganism of claim 2 in the presence of a carbon source under conditions effective to produce a fatty ester composition comprising beta-hydroxy fatty esters in a culture.

4. The method of claim 3, wherein the beta-hydroxy fatty esters include beta-hydroxy methyl esters or beta-hydroxy ethyl esters.

5. The method of claim 4, wherein the conditions include the presence of methanol.

6. The method of claim 5, wherein the methanol is included in or added to the culture.

7. The method of claim 5, wherein the methanol is produced by the recombinant microorganism.

8. The method of claim 4, wherein the composition comprises beta-hydroxy ethyl esters.

9. The method of claim 8, wherein the conditions include the presence of ethanol.

10. The method of claim 9, wherein the ethanol is included in or added to the culture.

11. The method of claim 9, wherein the ethanol is produced by the recombinant microorganism.

12. The method of claim 3, wherein the microorganism is a bacterium.

13. The method of claim 3, wherein the microorganism is a yeast.

14. The method of claim 12, wherein the bacterium is of the species Escherichia coli.

15. The method of claim 3, wherein the thioesterase has at least 90% amino acid sequence identity to a thioesterase encoded by tes A or ′tesA from E. coli.

16. The method of claim 3, wherein the acyl-CoA synthase has at least 90% amino acid sequence identity to an acyl-CoA synthase encoded by fadD from E. coli.

17. The method of claim 3, wherein the recombinant microorganism is engineered to have reduced expression of a fatty acid degradation enzyme or an outer membrane protein receptor.

18. The method of claim 17, wherein the microorganism is of the species Escherichia coli and has reduced expression of fhuA.

19. The method of claim 17, wherein the microorganism is of the species Escherichia coli and has reduced expression of fadE.

20. The method of claim 3, wherein the thioesterase is a tesA engineered to have enhanced ability to use acyl-ACP as a substrate, relative to a corresponding wild-type tesA.

21. The method of claim 3, wherein the polynucleotide sequence encoding the polypeptide having the ester synthase (EC 2.3.1.75) activity is located on a plasmid.

22. The method of claim 3, wherein the polynucleotide encoding a polypeptide having the ester synthase (EC 2.3.1.75) activity is integrated into a chromosome of the microorganism.

23. The method of claim 3, wherein the composition has a fatty acid methyl ester or fatty acid ethyl ester titer of at least about 5 g/L.

24. The method of claim 23, wherein the composition has a fatty acid methyl ester titer of at least about 45 g/L.

25. The method of claim 23, wherein beta-hydroxy methyl esters or beta-hydroxy ethyl esters comprise at least 5% of the total methyl or ethyl esters.

26. The method of claim 4, further comprising separating fatty acid methyl esters or fatty acid ethyl esters from the culture to form an enriched fatty acid methyl ester or fatty acid ethyl ester fraction.

27. The method of claims 26, further comprising polishing the enriched fatty acid methyl ester or fatty acid ethyl ester fraction.

28. The method of claim 3, wherein the composition has a percentage of total fatty acid methyl esters as follows:

| | |
|---|---|
| Methyl octanoate (C8:0) | 0-5% |
| Methyl decanoate (C10:0) | 0-2%; |
| Methyl dodecanoate (C12:0): | 0-5%; |
| Methyl dodecenoate (C12:1): | 0-10%; |
| Methyl tetradecanoate (C14:0): | 30-50%; |
| Methyl 7-tetradecenoate (C14:1): | 0-10%; |
| Methyl hexadecanoate (C16:0): | 0-15%; |
| Methyl 9-hexadecenoate (C16:1): | 10-40%; and |
| Methyl 11-octadecenoate (C18:1): | 0-15%[[;]]. |

29. The method of claim 28, wherein the fatty acid methyl esters comprise at least 5% of beta-hydroxyl esters.

30. (canceled)

31. The method of claim 3, further comprising purifying the beta-hydroxy fatty esters to form a beta-hydroxy fatty ester enriched fraction.

32. The method of claim 3, wherein the carbon source is biomass.

33. A composition produced by the method of claim 3.

34. A biodiesel composition comprising a fatty acid methyl ester composition wherein a percentage of total fatty acid methyl esters is as follows:

| | |
|---|---|
| Methyl octanoate (C8:0) | 0-5%; |
| Methyl decanoate (C10:0) | 0-2%; |
| Methyl dodecanoate (C12:0): | 0-5%; |
| Methyl dodecenoate (C12:1): | 0-10%; |
| Methyl tetradecanoate (C14:0): | 30-50%; |
| Methyl 7-tetradecenoate (C14:1): | 0-10%; |
| Methyl hexadecanoate (C16:0): | 0-15%; |
| Methyl 9-hexadecenoate (C16:1): | 10-40%; and |
| Methyl 11-octadecenoate (C18:1): | 0-15% |

with at least 5% of the fatty acid methyl esters comprising beta-hydroxy methyl esters.

35. The method of claim 4, wherein the composition comprises beta-hydroxy methyl esters.
